(19) United States
(12) Patent Application Publication
(10) Pub. No.: US 2021/0027888 A1
(43) Pub. Date: Jan. 28, 2021

(54) ARTIFICIAL INTELLIGENT PLATFORM FOR COLLABORATING, AUTOMATING AND ORGANIZING DRUGS/MEDICAL/HEALTH-INFORMATION BETWEEN STAKEHOLDERS IN THE PHARMACEUTICAL/HEALTHCARE INDUSTRIES

(71) Applicants: KIRAN SINGH BHATIA, Mississauga (CA); ISHAN ALOIS SINGH BHATIA, Mississauga (CA); ARJAN FELIX SINGH BHATIA, Mississauga (CA); USHVINDER SINGH BHATIA, Mississauga (CA)

(72) Inventors: KIRAN SINGH BHATIA, Mississauga (CA); ISHAN ALOIS SINGH BHATIA, Mississauga (CA); ARJAN FELIX SINGH BHATIA, Mississauga (CA); USHVINDER SINGH BHATIA, Mississauga (CA)

(21) Appl. No.: 16/934,301

(22) Filed: Jul. 21, 2020

Related U.S. Application Data
(60) Provisional application No. 62/877,381, filed on Jul. 23, 2019.

(51) Int. Cl. G16H 50/20; G06N 5/04; G16H 20/10; G16H 80/00; G16H 40/20; G06Q 10/10; G06Q 30/06; G06Q 30/08; G16H 50/70; G16H 70/40; G16H 10/60; G06F 21/62; H04L 29/06; H04L 12/58; H04L 12/18; H04N 7/15; G08B 21/24

(52) U.S. Cl. CPC G16H 50/20; G16H 40/67; G16H 20/10; G16H 80/00; G16H 40/20; G06Q 10/109; G06Q 30/0605; G06Q 30/0623; G06Q 30/0633; G06Q 30/0631; G06Q 30/08; G16H 50/70; G16H 70/40; G16H 10/60; G06F 21/6245; H04L 63/04; H04L 51/046; H04L 12/1895; H04L 12/1813; H04N 7/15; G08B 21/24; G06N 5/043

(57) ABSTRACT

Exemplary embodiments of the present disclosure are directed towards an artificial intelligence platform and computer implemented method for collaborating, automating and organizing drugs/medical/consultations/health information & Services between stakeholders in the pharmaceutical/healthcare industries. This also extends to professionals in the veterinary space such as veterinarians and animal specialists. The medical information generation module comprising: prescription fulfillment module, collaboration module, health assistant module, analytics module, virtual pharmacy & consultation module, and prescription auction module. The prescription fulfillment module is configured to use medical information collected from a database and, with the use of artificial intelligence, display medical information data and additional supplements & services suggestions on computing devices based on users. The collaboration module is configured to collaborate users with each other and streamline medical communication. It can be configured to generate real time alerts to proper users on the computing devices and also leverage video conferencing from the video consultation function.

### CROSS-REFERENCE TO RELATED APPLICATIONS

This patent application claims priority benefit of U.S. Provisional Patent Application No. 62/877,381, entitled “ARTIFICIAL INTELLIGENT PLATFORM FOR COLLABORATING, AUTOMATING AND ORGANIZING DRUGS/MEDICAL/HEALTH-INFORMATION BETWEEN STAKEHOLDERS IN THE PHARMACEUTICAL/HEALTHCARE INDUSTRIES”, filed on 23 Jul. 2019. The entire contents of the patent application is hereby incorporated by reference herein in its entirety.

### COPYRIGHT AND TRADEMARK NOTICE

This application includes material which is subject or may be subject to copyright and/or trademark protection. The copyright and trademark owner(s) has no objection to the facsimile reproduction by any of the patent disclosure, as it appears in the Patent and Trademark Office files or records, but otherwise reserves all copyright and trademark rights whatsoever.

### TECHNICAL FIELD

The present disclosure generally relates to the field of electronic health history information organization ecosystem. More particularly, the present disclosure relates to an artificial intelligence platform and a computer implemented method for collaborating, automating and organizing drugs/medical/consultations/health information & Services between stakeholders in the pharmaceutical/healthcare as well as in the veterinary industries.

### BACKGROUND

Prescriptions and records are largely paper-based, particularly in health centres where most care is delivered. The paper based process initiates with collecting medical history and other relevant healthcare information from patients on paper through registration forms provided to the patients when they present themselves for care. In addition to being paper-based, the collection of results in inaccurate and incomplete information because the patients forget to give necessary information and they hurry to finish the formalities. A patient's medical history is valuable in order to recognize genetic disorders that result in predictable complications. It is very difficult to eliminate the tedious hard copy exchange and execution of prescriptions by the physicians.

Additionally, even though most physicians ask for relevant personal medical history from their patients, as medical treatments become more complex, the demand for better access to all relevant personal and family medical records will only increase. Some existing solutions are available to users such as patients, physicians, and pharmacists to communicate with each other but those existing solutions cannot provide real time alerts and information to the users analysed by artificial intelligence models. The existing solutions do not provide advice on drug interactions, supplement suggestions, and dietary recommendations. Many conventional applications are configured to manage the collection, consolidation, and maintenance of a patient's medical data but, the conventional solutions do not provide a sufficient basis for a health adviser to review background data in a quick comprehensive manner. Additionally, the conventional solutions can't manage patient's health condition information and also generate appropriate prescriptions using the personal health history. There is no existing capabilities for a patient to procure bids from a large number of medical service providers/pharmacies for his/her prescription or needs.

In the light of aforementioned discussion, there exists a need for systems with novel methodologies that would overcome or ameliorate the above mentioned disadvantages.

### BRIEF SUMMARY

The following presents a simplified summary of the disclosure in order to provide a basic understanding to the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

Exemplary embodiments of the present disclosure are directed towards an artificial intelligence platform and a computer implemented method for collaborating, automating and organizing drugs/medical/health information between stakeholders in the pharmaceutical/healthcare and veterinary space industries.

An objective of the present disclosure is directed towards eliminating the tedious hard copy exchange and execution of prescriptions by automating communication and fulfillment between the users.

Another objective of the present disclosure is directed towards generating continuous real time alerts to the users for reminders such as prescription refills, key health check recommendations, due lab work, auction alerts, etc.

Another objective of the present disclosure is directed towards providing ecosystem for various stakeholders to collaborate in the pharmaceutical industry. For example, patients, pharmacists, doctors, medical practitioners, drug manufacturers, supplement providers, veterinarians as well as animal doctors and specialists can collaborate and transact efficiently and cost effectively.

Another objective of the present disclosure is directed towards eliminating the tedious hard copy exchange and fulfillment of prescriptions by automating communication and fulfillment between the doctor, pharmacist, and the patient, including integration with pharmacy, doctor, medical and hospital systems.

Another objective of the present disclosure is directed towards a real time alert system as applicable to patients, pharmacists, and doctors for reminders such prescription refills, key health check recommendations, due blood work, and so forth.

Another objective of the present disclosure is directed towards deep learning models to advise of drug interactions, supplement suggestions, and dietary recommendations.

Another objective of the present disclosure is directed towards advising drug interactions, supplement suggestions, and dietary recommendations.

Another objective of the present disclosure is directed towards providing drug cross-border equivalence and marketplace where all the users can transact.

Another objective of the present disclosure is directed towards health management (past, current advices, for e.g.) to assist in monitoring and maintenance of health history and prescription. Including direct communication like online video conferencing.

Another objective of the present disclosure is directed towards enabling the patients to upload prescription for an auction post.

Another objective of the present disclosure is directed towards generating automatic bids based on previous settings and bid information.

Another objective of the present disclosure is directed towards filtering auction posts based on various settings such as distance, inventory, services, price, etc.

Another objective of the present disclosure is directed towards enabling the pharmacies to place bids on auction posts, competing for lower prices.

### BRIEF DESCRIPTION OF DRAWINGS

Other objects and advantages of the present invention will become apparent to those skilled in the art upon reading the following detailed description of the preferred embodiments, in conjunction with the accompanying drawings, wherein like reference numerals have been used to designate like elements, and wherein:

FIG. 1 is a block diagram depicting an artificial intelligence platform for collaborating, automating and organizing drugs/medical/health information between stakeholders in the pharmaceutical/healthcare and veterinary industries, in accordance with one or more exemplary embodiments.

FIG. 2 is a block diagram depicting the medical information generation module 114 as shown in FIG. 1, in accordance with one or more exemplary embodiments.

FIG. 3 is a block diagram depicting a prescription fulfillment module 203 as shown in FIG. 2, in accordance with one or more exemplary embodiments.

FIG. 4 is a block diagram depicting a collaboration module 205 as shown in FIG. 2, in accordance with one or more exemplary embodiments.

FIG. 5 is a block diagram depicting a health assistant module 207 as shown in FIG. 2, in accordance with one or more exemplary embodiments.

FIG. 6 is a block diagram depicting a analytics module 211 as shown in FIG. 2, in accordance with one or more exemplary embodiments.

FIG. 7 is a block diagram depicting a marketplace module 213 as shown in FIG. 2, in accordance with one or more exemplary embodiments.

FIG. 8 is a block diagram depicting a virtual pharmacy and consultation module 217 as shown in FIG. 2, in accordance with one or more exemplary embodiments.

FIG. 9 is a block diagram depicting a prescription auction module 219 as shown in FIG. 2, in accordance with one or more exemplary embodiments.

FIG. 10 is an example block diagram depicting an end to end pharmaceutical ecosystem, in accordance with one or more exemplary embodiments.

FIG. 11 is an example flow diagram depicting a health Wize SAAS platform, in accordance with one or more exemplary embodiments.

FIG. 12 is an example block diagram depicting the prescription fulfillment module 203, in accordance with one or more exemplary embodiments.

FIG. 13 is an example block diagram depicting the collaboration module 205, in accordance with one or more exemplary embodiments.

FIG. 14 is an example block diagram depicting the health assistant module 207, in accordance with one or more exemplary embodiments.

FIG. 15 is an example block diagram depicting the drug related analytics module 211, in accordance with one or more exemplary embodiments.

FIG. 16 is an example block diagram depicting the marketplace module 213, in accordance with one or more exemplary embodiments.

FIG. 17 is an example block diagram depicting the virtual pharmacy and consultation module 217, in accordance with one or more exemplary embodiments.

FIG. 18 is an example block diagram depicting the prescription auction module 219, in accordance with one or more exemplary embodiments.

FIG. 19 is a flow chart depicting an exemplary method for collaborating and organizing medical information between multiple users in the pharmaceutical/healthcare industries, in accordance with one or more exemplary embodiments.

FIG. 20 is a flow chart depicting an exemplary method for collaborating the first user with the second user and the third user, in accordance with one or more exemplary embodiments.

FIG. 21 is a flow chart depicting an exemplary method for collaborating the second user with the first user and the third user, in accordance with one or more exemplary embodiments.

FIG. 22 is a flow chart depicting an exemplary method for collaborating the fourth user, fifth user with the first user, the second user, and the third user, in accordance with one or more exemplary embodiments.

FIG. 23 is a flow chart depicting an exemplary method for collaborating the third user with other users, in accordance with one or more exemplary embodiments.

FIG. 24 is a block diagram depicting the details of the computing device in which various aspects of the present disclosure are operative by execution of appropriate software instructions.

### DETAILED DESCRIPTION

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced item. Further, the use of terms “first”, “second”, and “third”, and the like, herein do not denote any order, quantity, or importance, but rather are used to distinguish one element from another.

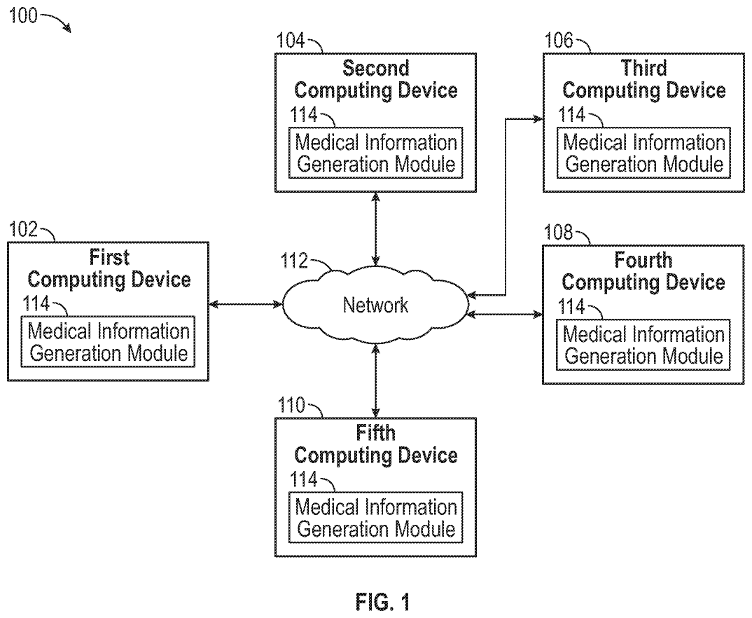

Referring to FIG. 1 is a block diagram 100, depicting an artificial intelligence platform for collaborating, automating and organizing medical information between stakeholders in the pharmaceutical/healthcare and veterinary industries, in accordance with one or more exemplary embodiments. The artificial intelligence platform 100 includes a first computing device 102, a second computing device 104, a third computing device 106, a fourth computing device 108, and a fifth computing device 110, and a network 112. The term computing devices 102, 104, 106, 108, 110 are used herein to refer to any device with processing capability such that it can execute instructions. Those skilled in the art will realize that such processing capabilities are incorporated into many different devices and therefore the term computing device 102, 104, 106, 108, 110 each include smart phones, PCs, servers, tablet computers, personal digital assistants and many other devices. The computing devices 102, 104, 106, 108, 110 may include a medical information generation module 114 which is accessed as mobile applications, web applications, software that offers the functionality of accessing mobile applications, and viewing/processing of interactive pages, for example, are implemented in the computing devices 102, 104, 106, 108, 110 as will be apparent to one skilled in the relevant arts by reading the disclosure provided herein. The medical information generation module 114 may be downloaded from the cloud server (not shown). For example, the medical information generation module 114 may be any suitable applications downloaded from, GOOGLE PLAY® (for Google Android devices), Apple Inc.'s APP STORE® (for Apple devices, or any other suitable database). In some embodiments, the medical information generation module 114 may be software, firmware, or hardware that is integrated into the computing devices 102, 104, 106, 108 and 110.

The network 112 may include but not limited to, an Internet of things (IoT network devices), an Ethernet, a wireless local area network (WLAN), or a wide area network (WAN), a Bluetooth low energy network, a ZigBee network, a WIFI communication network e.g., the wireless high speed internet, or a combination of networks, a cellular service such as a 4G (e.g., LTE, mobile WiMAX) or 5G cellular data service, a RFID module, a NFC module, wired cables, such as the world-wide-web based Internet, or other types of networks may include Transport Control Protocol/Internet Protocol (TCP/IP) or device addresses (e.g. network-based MAC addresses, or those provided in a proprietary networking protocol, such as Modbus TCP, or by using appropriate data feeds to obtain data from various web services, including retrieving XML data from an HTTP address, then traversing the XML for a particular node) and so forth without limiting the scope of the present disclosure.

The medical information generation module 114 may be a SAAS cloud platform which provides an artificial intelligence for the multiple stakeholders. The multiple stakeholders/users may include a first user, a second user, a third user, a fourth user, and a fifth user. The first user may include but not limited to, a patient, a friend, a relative, an employee, and so forth. The second user may include, but not limited to, a doctor, a medical practitioner, a physician, a caregiver, psychologist, psychiatrists, veterinarians, physiotherapists, life coaches and so forth. The third user may include, but not limited to, a pharmacist, a pharmacy, a druggist, a chemist, a pharmacologist, a nurse, and so forth. The fourth and fifth users may include, but not limited to, drug manufacturers, supplement providers, supplement/natural medicine providers, health care organizations, and so forth. The multiple stakeholders may collaborate and transact efficiently and cost effectively through the computing devices 102, 104, 106, 108, and 110. The medical information generation module 114 may be configured to eliminate the tedious hard copy exchange and execution of prescriptions by automating and streamlining communication and fulfillment between the users. This includes integration with software systems that are used by these practitioners (doctors, pharmacists, veterinarians, psychiatrist, life coaches and so forth). The medical information generation module 114 comprises implementation of deep learning techniques to advice regarding drug interactions, supplement suggestions, and dietary recommendations. The medical information generation module 114 may also be configured to provide drug cross-border equivalence and marketplace where all users may transact. The medical information generation module 114 may be a long-term sustainable model to keep enhancing usage, functionality, and market penetration. The medical information generation module 114 may be configured to enable the users to expose to different features based on what they value. The medical information generation module 114 may be configured to provide medical information such as past medical history, family medical history, medication history, personal and lifestyle history, psychological analysis, vital signs (heart rate, respiration, blood pressure, temperature, oxygen saturation, for e.g.), electrocardiogram (EKG or ECGs), and other labs such as drug level, bacteria in urine, bacteria in the blood, activities of the individual, glucose levels, and so forth. The medical information is also extended to animals, providing similar metrics and information but also including animal-specific metrics such as breed, diseases, castrations, vaccinations and so forth.

The medical information generation module 114 may be configured to enable the users (for example, patients) to fulfil the prescriptions with total automation and peace of mind. The users may have the ability for them to keep track of the health as time progresses. The medical information generation module 114 may also be configured to give suggestions from artificial intelligence models that are based on the prescription history, and gives them reminders regarding when to refill and when to take medication (along with dose amount). The users (for example, patient) may also have an ability to easily reach out to other users (pharmacies, doctors, clinics, veterinarians, specialists). For example, the patients reach the doctors through the medical information generation module 114 which provides communication channels. The communication channels may include but not limited to, a chat communication channel, video communication channel, an audio communication channel, and so forth.

For the first user and the second user, they may have access to the online marketplace module 213 to purchase and browse the medical products. Those medical products may be given by the fourth and fifth users. For the fourth and fifth users, they may have additional functionality to upload and modify which medical products get posted on the medical information generation module 114. The fourth and fifth users may have access to statistics such as user traffic and clicks.

The medical information generation module 114 may be configured to provide the users (for example, doctors such as immunologists, anesthesiologists, cardiologists, colon and rectal surgeons, critical care medicine specialists, family physicians, veterinarians, specialists, psychiatrist, life coaches gastroenterologists, allelopathy, ayurvedic, homeopathy, and other natural medicines, and so forth) with an easy way of keeping track of patient prescriptions. Instead of manually writing prescriptions and giving it to the users, they may have the facility of sending the prescription straight to the pharmacy of choice. This not only saves time, but makes it easier and cost effective for the users.

The medical information generation module 114 may be configured to provide the users (for example, pharmacists) with efficient, cost effective prescription handling as well as direct patient collaboration with use of artificial intelligence, advanced chatbots and other technologies. By using the medical information generation module 114, pharmacists may also prepare orders ahead of time and may constantly keep track of the inventory.

The medical information generation module 114 may be configured to provide subscription services to the users of the computing devices 102, 104, 106, 108, 110. The medical information generation module 114 may also be configured to allow the users to advertise the products to other users, a revenue stream may be generated by artificial intelligence models based on views and on clicks of advertisements. There is revenue sharing, whenever the user decides to purchase a drug or supplement through the medical information generation module 114 the user (for example, admin) may receive a percentage of the transaction. As the project progresses there may be more opportunities to remove, add and refine current revenue streams.

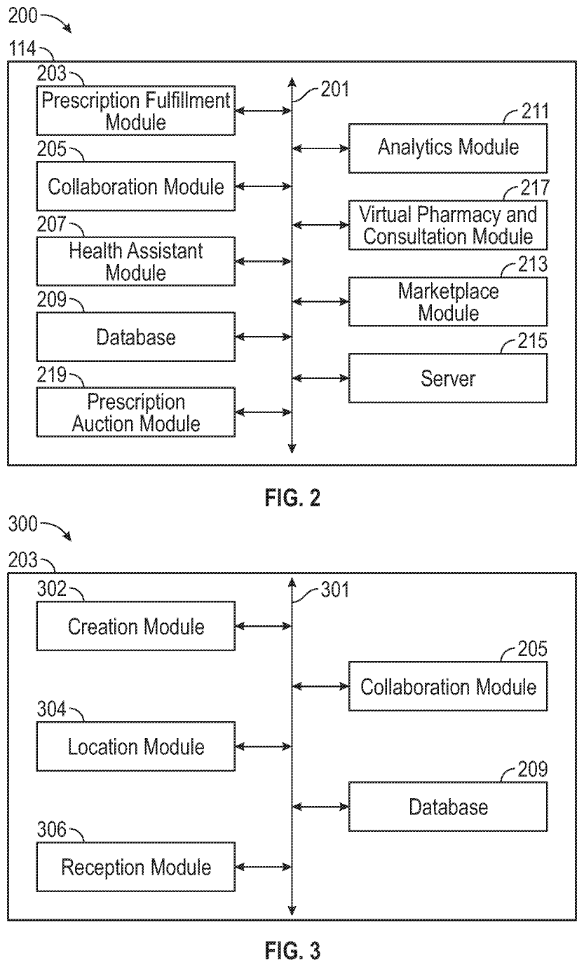

Referring to FIG. 2 is a block diagram 200 depicting the medical information generation module 114 as shown in FIG. 1, in accordance with one or more exemplary embodiments. The medical information generation module 114 comprises a bus 201, a prescription fulfillment module 203, a collaboration module 205, a health assistant module 207, a database 209, an analytics module 211, a marketplace module 213, a cloud server 215, and a virtual pharmacy and consultation module 217, and a prescription auction module 219. The bus 201 may include a path that permits communication among the modules of the medical information generation module 114. The database 209 may be configured to store, retrieve, process, analyze, and/or generate medical information to be provided to the computing devices 102, 104, 106, 108, 110. The term “module” is used broadly herein and refers generally to a program resident in the memory (not shown) of the computing device 102 or 104 or 106 or 108 or 110.

The prescription fulfillment module 203 may be configured to use the medical information from the database 209 but displays the medical information differently based on the user. For example, the prescription fulfillment module 203 is configured to display the prescriptions that are currently in use, what prescriptions are completed, pending auto-renewals where there is need to have access, and to visualize other information based on prescription data. The prescription fulfillment module 203 is also configured to display the list of patients to the doctors that they are dealing with along with each patient's current and completed prescriptions. The doctors may also access to pharmacy locations to be able to automatically send the prescription to the respective locations. The prescription fulfillment module 203 is also configured to display the list of pending prescriptions to the pharmacists and also display a log of all the prescriptions that have been fulfilled in the past. The prescription fulfillment module 203 may be configured to show the user the medical information that is relevant to the prescriptions. For example, the medical information generation module 114 can provide a button to the user, that button says “show prescription drug information”.

The medical information generation module 114 extends to veterinarians. The first user may upload postings regarding pets, for example, the problem they are having or disease they have. Based on the postings, the medical information generation module 114 may then match the correct veterinarian to the post/patient. Once a match is taken place then the first user may request a certain medication or the specialist may arrange an appointment to diagnose the pet. Using the virtual pharmacy and consultation module 217, diagnosis may also take place remotely, through a video conference. Once diagnosis has taken place, the first user may then request the pickup or delivery of the medication through the prescription fulfillment module 203.

The collaboration module 205 may be configured to collaborate multiple users with each other with the use of chats and alert systems. The collaboration module 205 may also be configured to generate real time alerts to the proper users for reminders such as prescription refills, key health check recommendations, due lab work, and so forth. For example, the doctors would not want notifications on product special offers. Another example is the doctors and pharmacists would not want notifications on when the patient must take the medication. As of now we have different types of alerts for the different users and they have the ability to enable/disable these notifications based on their preference. The collaboration module 205 may also be configured to generate real time alerts to the users if there are conflicts between certain drugs in their prescriptions. For example, if there are conflicts between certain drugs in the prescriptions, an alert can be sent via the collaboration module 205 directly to the patient.

The health assistant module 207 may be configured to enable the user to access a personal chatbot. The chatbot (not shown) may be configured to provide the medical information comes from the analytics module 211. The chatbot may also show the user access to the prescriptions for example (User: “Show me my current prescription”, Chatbot:“OK, here it is: . . . ”). The chatbot may also display product and offers from the collaboration module 205 and the marketplace module 213. From the collaboration module 205, the chatbot may display custom recommendations given by the users. The health assistant module 207 may be configured to provide the helpful tips that may improve the user's daily health. The tips may include messages such as “Did you drink 7 glasses of water today?” the health assistant module 207 may also provide improved tips that are more customized to them. For example if the user has a prescription history of getting constant sore throats. The health assistant module 207 may be configured to recognize that and give user suggestions that may help the user with their sore throat. For example “Make sure to wear a scarf outside, it's cold!”. The chatbot is built on a base of artificial intelligence models. These models learn by using user data to help give accurate suggestions and recommendations. As users make decisions and ask the chatbot questions, the chatbot (and AI models) learns to give increasingly accurate information.

The analytics module 211 may be configured to enable the user to access to medical information (for example, drug information) and conflicts, cross-border drug conversations, and so forth. The analytics module 211 may also be configured to provide artificial intelligence image recognition/artificial intelligence image to text conversion to the users, drug information and correlations, and cross border drug conversions. The artificial intelligence image recognition may be used for the user to take a picture of the medicine or prescription they have and to automatically provide drug information, correlations, and cross border drug conversions. This may be used instead of the user manually typing in the medicine name and information (much faster to take the picture).

The marketplace module 213 may be configured to provide marketplace experiences to the users. The users may shop for the drugs and supplements through the marketplace module 213. The marketplace module 213 may be communicated with the health assistant module 207 configured to give the user personalized recommendations. Based on the usage of the marketplace (trends in what they typically buy), personalized offers may be displayed in the health assistant module 207. Using the artificial intelligence supplement suggestions, the user may get alerts on supplement suggestions.

The virtual pharmacy and consultation module 217 may be an interface that provides the third users to open up their own virtual pharmacy. The virtual pharmacy may be an online representation of a pharmacy that provides the first users with a place where they may purchase medicine and drug/supplements based on their prescription. The third users may not only upload the medicine they have available, but also may upload instructional videos based on the medicine that is prescribed. These videos may be viewed by the first user to get instructions on the dose amounts and information on the prescribed medication. The second users may also be able to look at the videos, give approval and provide comments in case they think additional clarification is required for specific cases. The virtual pharmacy and consultation module 217 may also be able to interact with other modules. The virtual pharmacy and consultation module 217 may also be configured to interact with the database 209 to provide the current patient prescription (also giving the users permission to buy certain medication if it's listed on their prescription). Using data, based on previous purchases, the artificial intelligence may give user suggestions on what medication to purchase from what pharmacy. The collaboration module 205 may be configured to take the information from the virtual pharmacy to provide the first users with alerts on product offers and medicine availability. The pharmacy locations interface provides the first users with a list of virtual pharmacies they may interact with. The virtual pharmacy and consultation module 217 may be configured to enable video conference between the multiple stakeholders. The virtual pharmacy and consultation module 217 may be configured to enable the video consultations on the first computing device 102 between the first user and the second user. The second users, the third users, the fourth users, the fifth users may also communicate through the medical information generation module 114 including payment handling. The second users, the third users, the fourth users, the fifth users are able to onboard on to the virtual pharmacy and consultation module 217, in order to host and handle their own sessions with the first users.

The prescription auction module 219 may be an interface that provides the ability of hosting and managing auction postings. The prescription auction module 219 may be configured to allow the first users to upload the prescriptions for an auction post. The prescription auction module 219 may be able to interact with other modules. The first user may set personal settings to control which third users, for example, pharmacies may view the post of auction. Settings may include clinic distance, delivery availability, services, stock and inventory, and the availability. When the prescriptions have uploaded on the interface, pharmacies may view the post, bid for the lower price, and then finally fulfil the prescription once they have won the auction. The prescription auction module 219 may also be configured to generate automatic bids based on previous settings and bid information. The collaboration module 205 may be configured to take the information from the prescription auction module 219 to provide the users with alerts on bids that they won. The first users may only view their own postings to maintain privacy and security, however the third users may view all postings. Once the timed auction posting is complete, the prescription fulfillment information may update, and the procedure is the same as a regular prescription upload.

The third users may filter which postings they want to view with settings such as distance, inventory and stock, services, price etc. The third user may keep bidding until the time is up and bid on multiple postings at once. After the bidding is complete, if the pharmacy has won the auction, the prescription may be placed in their fulfillment queue, and the procedure may be the same as a normal prescription request. The second users may also be able to view the pharmacy locations and may be able to rate on which pharmacies they approve. The second users may also give a rating based on pharmacy service and price. Finally, the fourth and fifth users may also view postings and offer package bundles associated with the medications that are prescribed. The fourth and fifth users may offer deals and suggestions that may go well with the current post. The first user may then have to approve or reject the supplement recommendations which furthers the learning of our artificial intelligence models.

Referring to FIG. 3 is a block diagram 300 depicting the prescription fulfillment module 203 as shown in FIG. 2, in accordance with one or more exemplary embodiments. The prescription fulfillment module 203 comprises the bus 301, a creation module 302, a location module 304, a reception module 306, the collaboration module 205, and the database 209. The bus 301 may include a path that permits communication among the modules of the prescription fulfillment module 203.

The creation module 302 may be configured to enable the second user to create the prescription for the first user. Here, the second user may include a doctor and the first user may include a patient. The creation module 302 may also be configured to enable the user to convert speech to text conversion using an artificial intelligence. For example the doctor can give speech through the creation module 302 for final creation of prescription. The final prescriptions from the doctor's computing device may be stored in the database 209. The location module 304 may be configured to enable the first user to select the location. For example, the patient selects the pharmacy locations through the location module 304. The reception module 306 may be configured to enable the third user to receive the prescription from the database 209. The reception module 306 may also be configured to enable the third user to log of pending and completed prescriptions. The creation module 302 may be communicated with the collaboration module 205 through the bus 301 for prescription shipment/fulfillment to the first user. The collaboration module 205 may be configured to generate the alert such as prescription auto-renewal alert to the third user. The collaboration module 205 may also be configured to integrate the first user, the second user, the third user through the computing devices 102, 104, 106.

When the third user (for example pharmacist) is fulfilling the prescription, the prescription fulfillment module 203 integrates with pharmacy management systems (PMS) and electronic medical records (EMR). An example of such a system is Kroll made by Telus Health. For example, when the pharmacist receives a prescription, they may have an option of uploading the data to the local PMS and automate the information entry into the local PMS for them. The pharmacist may then continue fulfilling the prescription through the third computing device 106. Once the prescription is fulfilled, the prescription fulfillment module 203 may notify the first user (for example patient) on the first computing device 102 and continue with the process of either pickup or delivery of the medication.

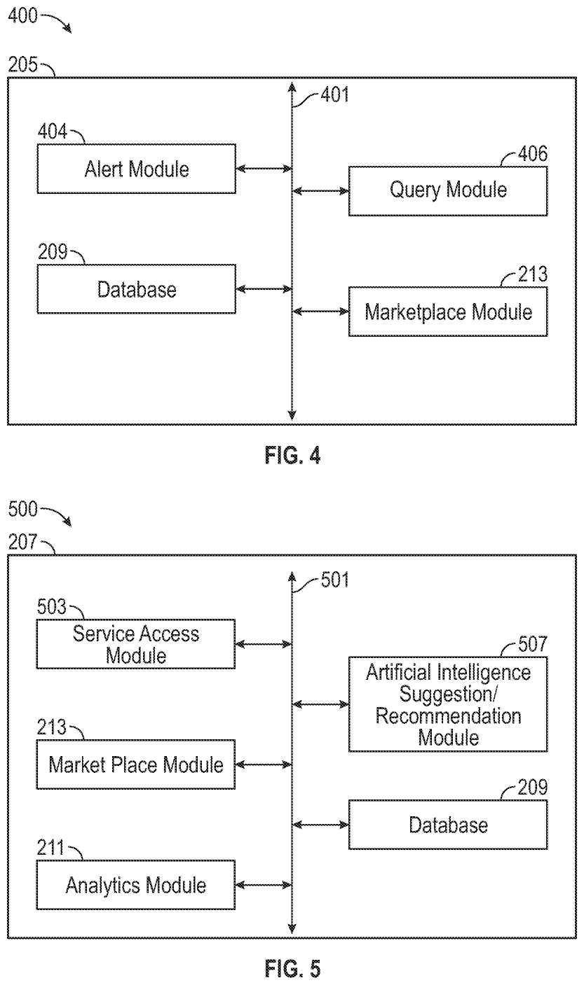

Referring to FIG. 4 is a block diagram 400 depicting the collaboration module 205 as shown in FIG. 2, in accordance with one or more exemplary embodiments. The collaboration module 205 comprises the bus 401, an alert module 404, a query module 406, the marketplace module 213, and the database 209. The bus 401 may include a path that permits communication among the modules of the collaboration module 205.

The alert module 404 may be configured to generate notifications to the first user for when to take medication. The alert module 404 may also be configured to generate notifications to the third user for pending prescriptions, refill remainders, important prescription information, and fulfillment/shipment notifications. Where the prescriptions are received from the database 209. The alert module 404 may also be configured to generate the alerts to the first user for the prescription refills and also generate the alerts for when the prescriptions fulfilled. For example, the alerts may include but not limited to, alerts for prescription refills, alerts for when to take the prescribed medicine, alerts on conflicting drugs within prescriptions, and so forth. The alert module 404 may also be configured to enable the second user to provide service alerts and refill requests to the first user. The query module 406 may be configured to provide the second user's questions to the first user and also enable the second user to give questions to the first user. The query module 406 may be configured to provide the questions in the form of a chat, where the first user (for example, patient) may message directly with the third user (pharmacist) or the second user (for example, doctor). The marketplace module 213 may be configured to generate product offers to the first user and the second user.

Referring to FIG. 5 is a block diagram 500 depicting the health assistant module 207 as shown in FIG. 2, in accordance with one or more exemplary embodiments. The health assistant module 207 comprises the bus 501, a service access module 503, an artificial intelligence suggestion/recommendation module 507, the analytics module 211, the marketplace module 213, and the database 209. The bus 501 may include a path that permits communication among the modules of the health assistant module 207.

The service access module 503 may be configured to assign access to the first user, the second user, and the third user. The service access module 503 may be configured to enable the first user, the second user, and the third user to access the prescriptions. Where the prescriptions are received from the database 209. The service access module 503 may also be configured to allow the users to access the other modules (for example, the analytics module 211, marketplace module 213) and services. The service access module 503 may be configured to associate the users with the user actions available to the users. The artificial intelligence suggestion/recommendation module 507 may be configured to communicate with the marketplace module 213 and the collaboration module 205 through the bus 501. The marketplace module 213 may be configured to generate advertisements to the first user and the second user through the collaboration module 205. The advertisements may include but not limited to, supplement advertisements, natural medicine advertisements, and so forth. The artificial intelligence suggestion/recommendation module 507 may be configured to generate suggestions/recommendations to the first user by the marketplace module 213 and the collaboration module 205. The suggestions/recommendations may include but not limited to, medicine replacement suggestions, health suggestions, personalized doctor recommendations, health facts and minor diagnosis, and so forth.

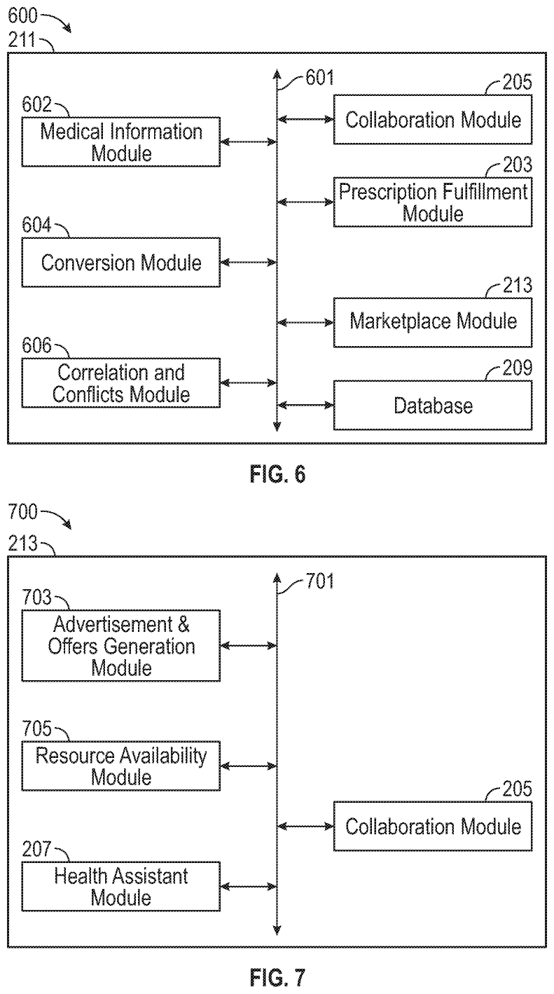

Referring to FIG. 6 is a block diagram 600 depicting the analytics module 211 as shown in FIG. 2, in accordance with one or more exemplary embodiments. The analytics module 211 comprises the bus 601, a medical information module 602, a conversion module 604, and a correlation and conflicts module 606, the prescription fulfillment module 203, the collaboration module 205, the marketplace module 213, and the database 209. The bus 601 may include a path that permits communication among the modules of the analytics module 211.

The medical information module 602 may be configured to provide the medical information and analysis to the users through the health assistant module 207. The medical information and analysis may include but not limited to, detailed drug and supplement/natural medicine information including chemicals and side effects. The medical information module 602 may be configured to communicate with the marketplace module 213. The conversion module 604 may be configured to convert the images to text using an artificial intelligence methodology. The uses of the converted text may include but not limited to, cross-border drug conversions, equivalent and similar medications in foreign countries, and so forth. The converted text may be transmitted to the computing devices 102/104/106/108/110 through the health assistant module 207. The correlation and conflict module 606 may be configured to correlate and conflict the medical information and transmit the alerts through the collaboration module 205. The correlation and conflict module 606 may be configured to find correlate and conflict medicines in the current or future prescriptions. Here, the medical information may include, but not limited to, prescription drug related information, detailed drug and supplement/natural medicine information including chemicals and side effects, patient allergy concerns, and so forth.

Referring to FIG. 7 is a block diagram 700 depicting the marketplace module 213 as shown in FIG. 2, in accordance with one or more exemplary embodiments. The marketplace module 213 comprises the bus 701, an advertisement and offer generation module 703, a resource availability module 705, the artificial intelligence suggestion/recommendation module 507, the health assistant module 207, and the collaboration module 205. The bus 701 may include a path that permits communication among the modules of the marketplace module 213.

The advertisement and offer generation module 703 may be configured to enable the fourth user and the fifth user to generate advertisements and offers to the first user and the second user through the health assistant module 207. The fourth user and the fifth user may include but not limited to, drug suppliers, supplement/natural medicine providers, health care organizations, and so forth. The advertisements and offers may include but not limited to, product advertisements, special offers on products, and so forth. The resource availability module 705 may be configured to enable the fourth user and the fifth user to provide the list of available resources. The list of available resources may include but not limited to, medicines, medical devices, drugs, and so forth. The artificial intelligence suggestion/recommendation module 507 may be configured to enable the fourth user and the fifth user to provide supplement suggestions and recommendations. The supplement suggestions and recommendations may be transmitted to the first computing device 102 through the collaboration module 205.

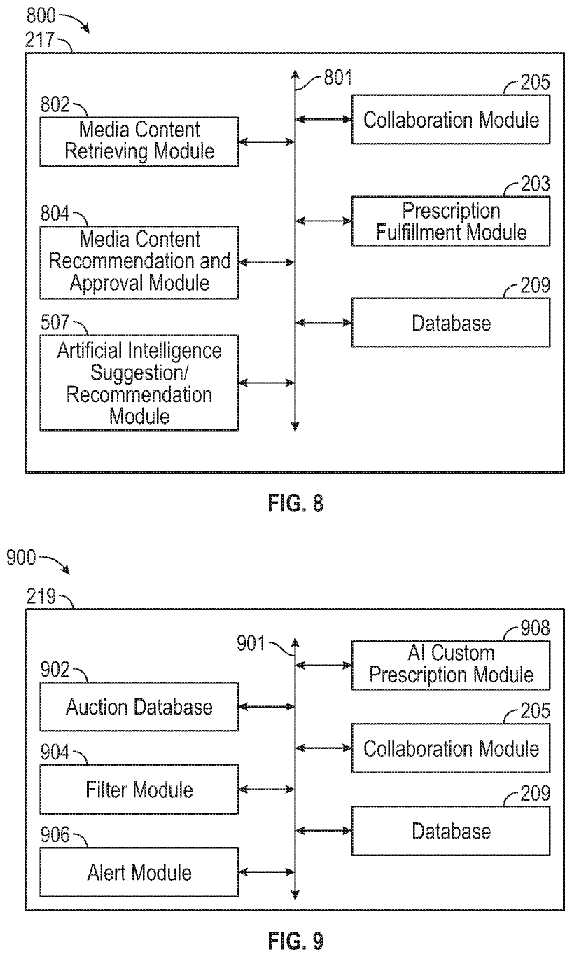

Referring to FIG. 8 is a block diagram 800 depicting the virtual pharmacy and consultation module 217 as shown in FIG. 2, in accordance with one or more exemplary embodiments. The virtual pharmacy and consultation module 217 provides a custom interface to run a virtual pharmacy. The virtual pharmacy and consultation module 217 comprises the bus 801, a media content retrieving module 802, a media content recommendation and approval module 804, the artificial intelligence suggestion/recommendation module 507, the collaboration module 205, the database 209, the prescription fulfillment module 203. The bus 801 may include a path that permits communication among the modules of the virtual pharmacy and consultation module 217. The prescription fulfillment module 203 may be configured to enable the first user to select the medicine to purchase. The media content retrieving module 802 may be configured to retrieve a media content to the first user on the first computing device 102 based on the pharmacy and prescription. The media content recommendation and approval module 804 may be configured to recommend the media content and also enables the second user to approve the media content on the second computing device 104. The media content recommendation and approval module 804 may also be configured to enable the second user on the second computing device 104 to rate, and comment on the media content. The artificial intelligence suggestion/recommendation module 507 may be configured to provide recommendations to the first user on the first computing device 102 through the collaboration module 205 based on previous purchases.

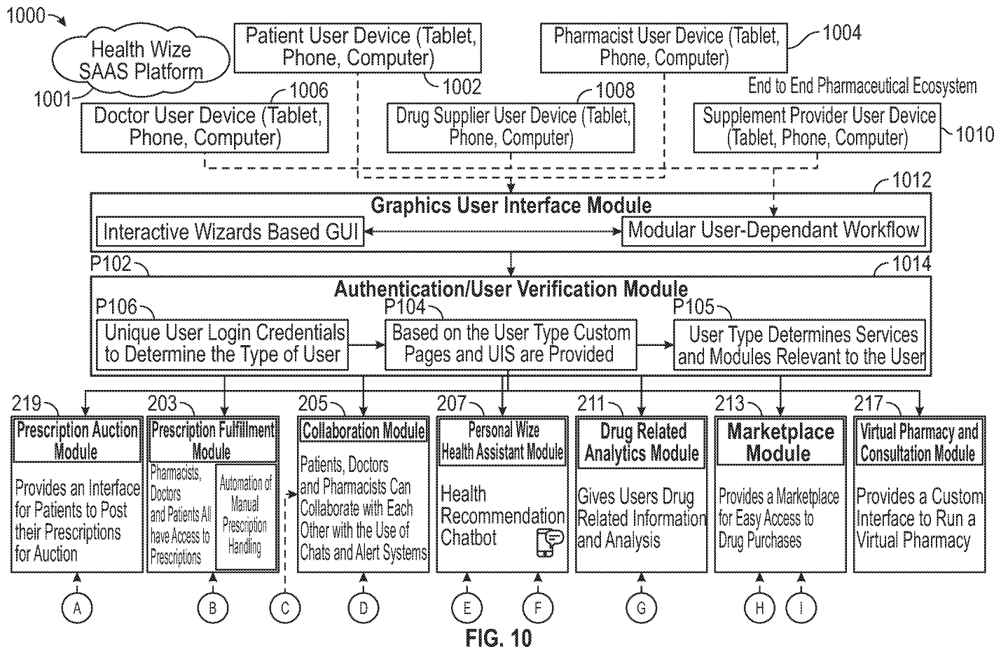

Referring to FIG. 9 is a block diagram 900 depicting the prescription auction module 219 as shown in FIG. 2, in accordance with one or more exemplary embodiments. The prescription auction module 219 comprises the bus 901, an auction database 902, a filter module 904, an alert module 906, an AI custom prescription module 908, the collaboration module 205, and the database 209. The bus 901 may include a path that permits communication among the modules of the prescription auction module 219.

The auction database 902 may be configured to store the data of current ongoing auctions as well as a log of previous completed auctions. Once auctions are completed, data may be transferred to the prescription fulfillment module 203, so that the request may be carried out like a regular prescription upload. There's also interaction with the collaboration module 205 to provide constant updates to all stakeholders for updates in auction and fulfillment status. The AI custom prescription module 908 may include a role in providing pharmacies with automatic bidding based on previous bids and other information such as stock, distance, and so forth. The AI custom prescription module 908 may also be configured to give drug manufacturers, automatic supplement packages using patient purchase data. These AI functionalities may help stakeholders with the auction process. The database 209 may be configured to supply prescription information to the prescription auction module 219.

The filter module 904 may be configured to enable the pharmacies to filter which postings they want to view with settings such as distance, stock, price, services and so forth. The third users may keep bidding until the time is up and bid on multiple postings at once. After the bidding is complete, if the pharmacy has won the auction, the prescription may be placed in their fulfillment queue, and the procedure may be the same as a normal prescription request. The filter module 904 may be configured to allow the user to set personal settings to control which pharmacies view the post. The alert module 906 may be configured to enable the third user and the first user to receive updates, alerts from posts that are being followed, receive alerts and updates from auction.

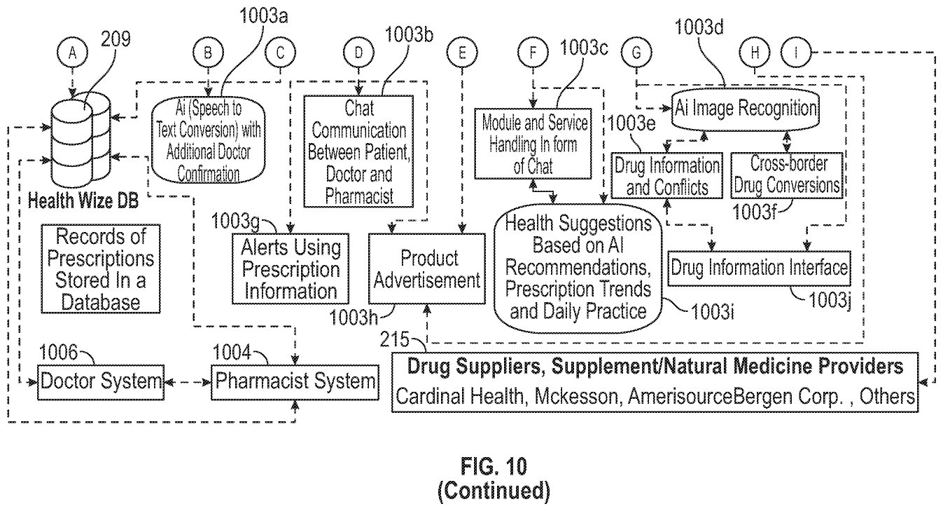

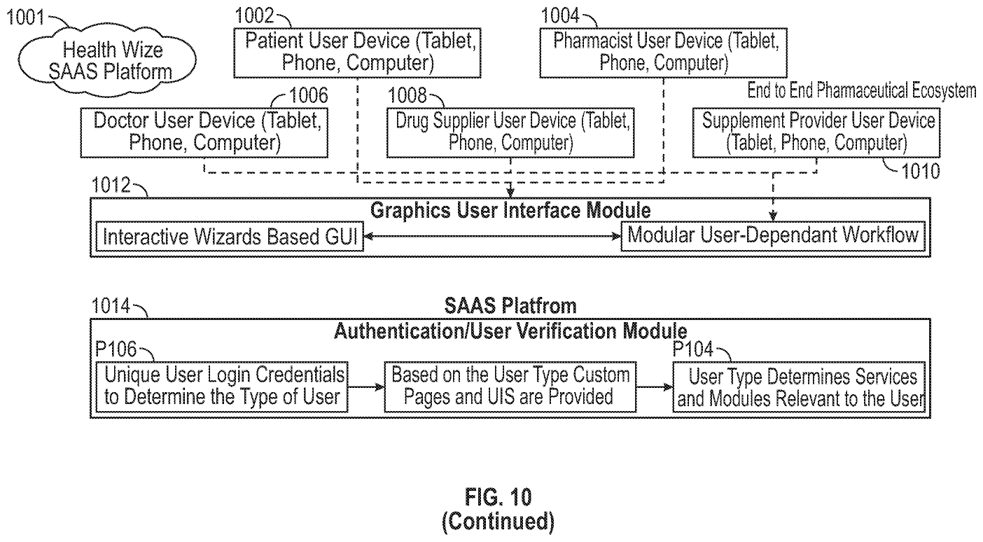

Referring to FIG. 10 is an example block diagram 1000 depicting an end to end pharmaceutical ecosystem, in accordance with one or more exemplary embodiments. The end to end pharmaceutical ecosystem 1000 includes a patient user device 1002, a pharmacist user device 1004, a doctor user device 1006, a drug supplier user device 1008, a supplement provider user device 1010, a graphical user interface module 1012, and an authentication/user verification module 1014. Based on the user type custom pages and UIs are provided on the devices 1002, 1004, 1006, 1008, 1010. Unique user login credentials to determine the type of user. The graphical user interface module 1012 may be configured to facilitate graphic user interface processing of the patient user device 1002, the pharmacist user device 1004, the doctor user device 1006, the drug supplier user device 1008, and the supplement provider user device 1010. The graphical user interface module 1012 includes an interactive wizards based GUI, and a modular user-dependent workflow. The authentication/user verification module 1014 may be configured to determine user type services and modules relevant to the user.

A Health Wize SAAS platform 1001 includes the prescription fulfillment module 203, the collaboration module 205, the personal wise health assistant module 207, the database 209, the drug related analytics module 211, the marketplace module 213, servers 215, the virtual pharmacy and consultation module 217, and the prescription auction module 219. The prescription fulfillment module 203 may be configured to enable pharmacists, doctors, patients to access prescriptions on the devices. The prescription fulfillment module 203 may also be configured to give speech to text conversion with additional doctor confirmation 1003a via the second computing device. The text may include medical related text. The prescription fulfillment module 203 may also be configured to provide automation of manual prescription handling. A chat block 1003b may be configured to provide chat communication between patient, doctor, and pharmacist. A module and service handling block 1003c may be configured to handle services and modules in form of chat. An AI image recognition block 1003d may be configured to provide image recognition/artificial intelligence image to text conversion to the users. The image recognition block 1003d may be communicated with a drug information and conflicts block 1003e and a cross-border drug conversions block 1003f. The term “module” is used broadly herein and refers generally to a program resident in the memory (not shown) of the computing device 1002 or 1004 or 1006 or 1008 or 1010. The drug information and conflicts block 1003e may be configured to provide drug information and conflicts. The cross-border drug conversions block 1003f may be configured to provide cross-border drug conversions. A drug information interface 1003j may be configured to interface between the drug information and conflicts block 1003e and the cross-border drug conversions block 1003f.

An alert block 1003g may be configured to generate alerts using prescription information. A product advertisement block 1003h may be configured to generate product advertisements. A health suggestion block 1003i may be configured to provide health suggestions based on AI recommendations, prescription trends, and daily practice. The collaboration module 205 may be configured to collaborate patients, doctors, and pharmacists with the use of chats and alert systems. The personal wise health assistant module 207 may be configured to provide a health recommendation chatbot. The health recommendation chatbot may give health suggestions based on AI recommendations, prescription trends and daily practice.

The drug related analytics module 211 may be configured to provide drug related information and analysis. The drug related information includes drug information and conflicts. The drug related analytics module 211 may also be configured to provide artificial intelligence image recognition/artificial intelligence image to text conversion to the users, drug information and correlations, and cross border drug conversations. The artificial intelligence image recognition may be used for the user to take a picture of the medicine they have and to automatically provide drug information, correlations and cross border drug conversions. The pharmacist system 1004 and the doctor system 1006 may be communicated with the database 209. The database 209 may include a Health Wize database configured to store records of prescriptions.

The marketplace module 213 may be configured to provide a marketplace for easy access to drug purchases. The servers 215 may include drug supplier servers, supplement/natural medicine provider servers, cardinal health servers, and so forth. The virtual pharmacy and consultation module 217 may be configured to provide a custom interface to run a virtual pharmacy. The prescription auction module 219 may be configured to provide an interface for patients to post their prescriptions for auction.

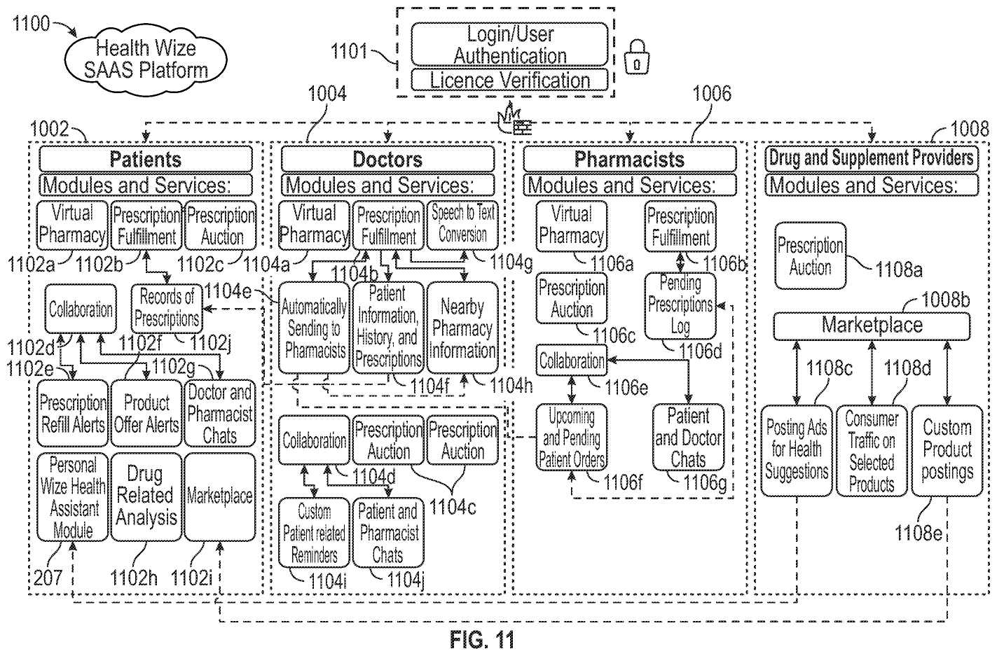

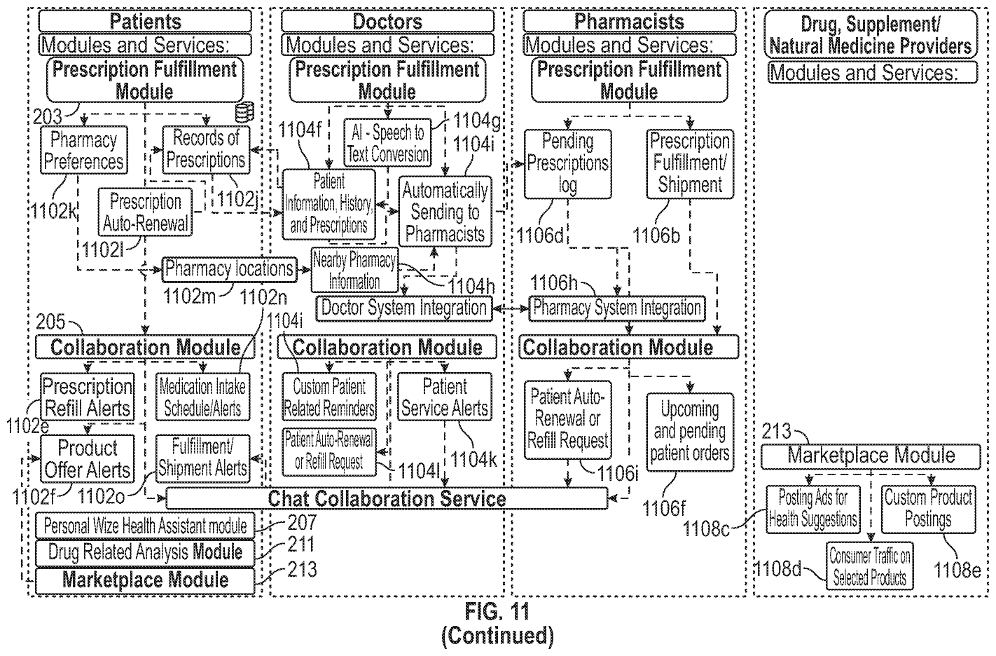

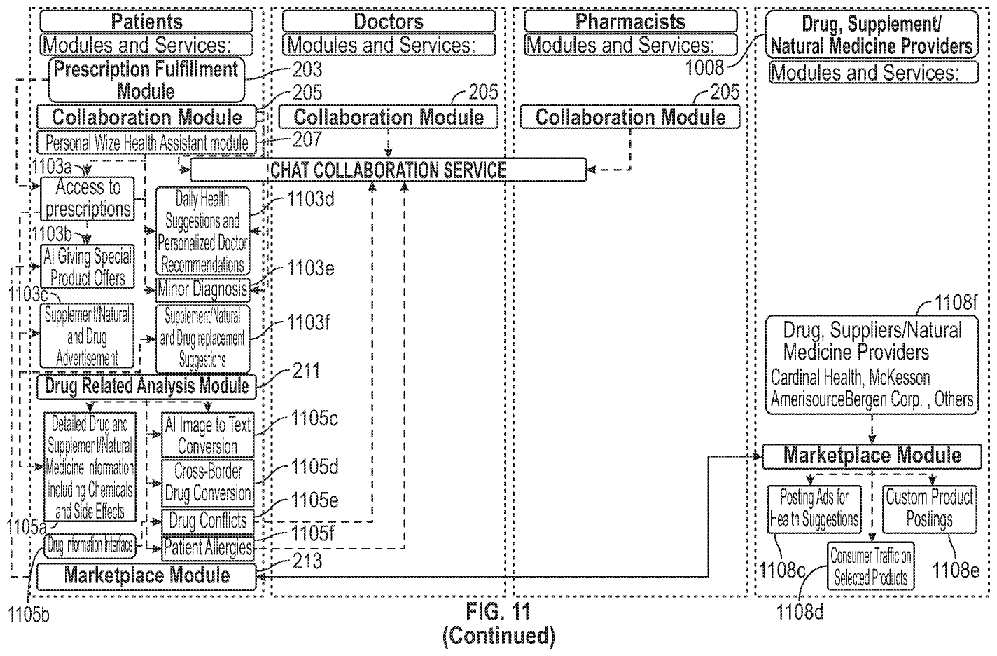

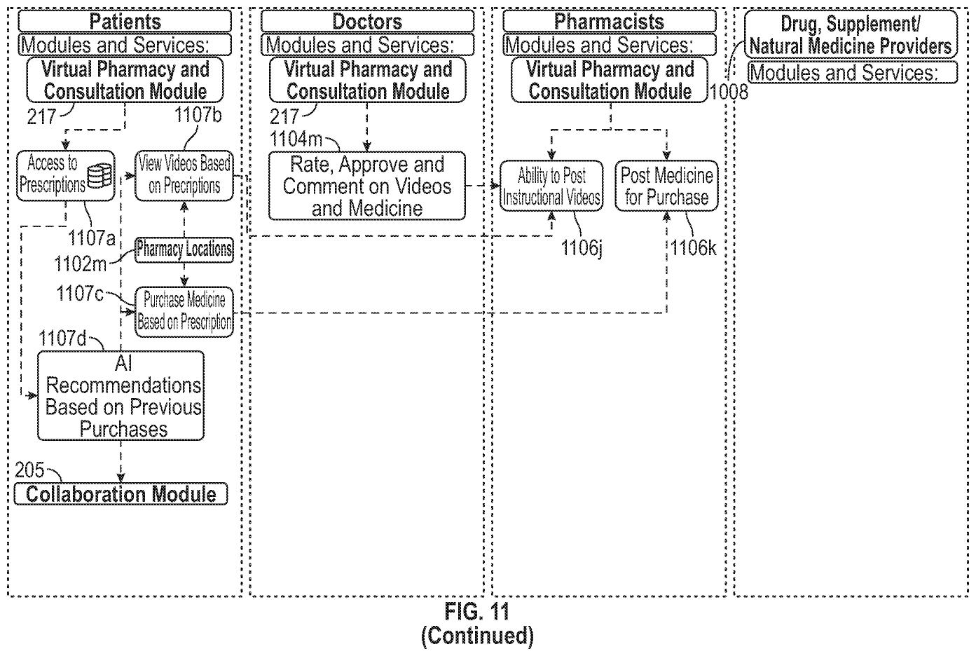

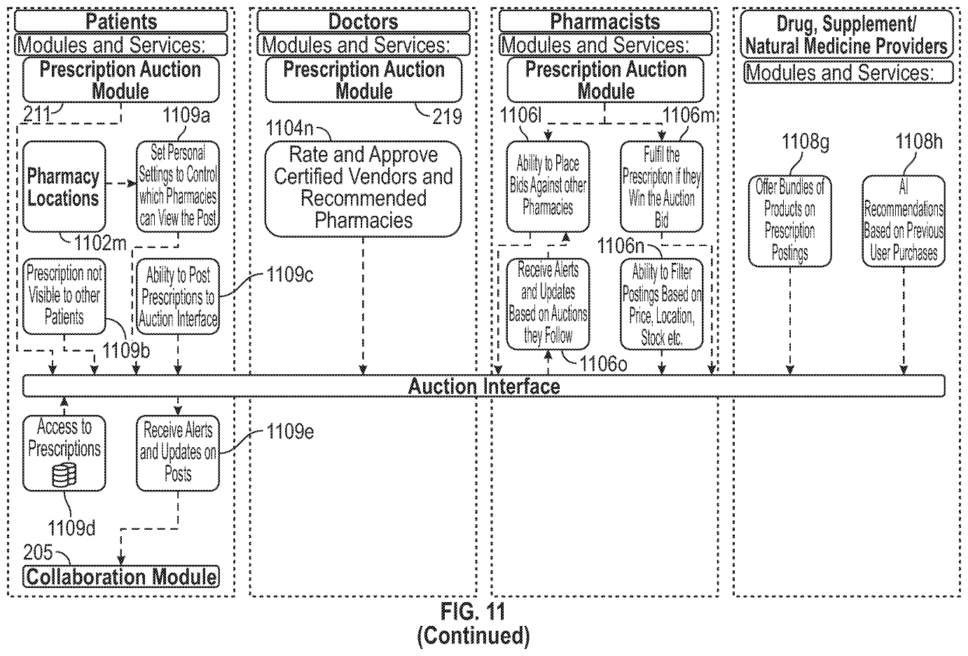

Referring to FIG. 11 is an example flow diagram 1100 depicting a Health Wize platform, in accordance with one or more exemplary embodiments. The Health Wize platform 1100 includes an authentication block 1101, the patient user device 1002, the pharmacist user device 1004, the doctor user device 1006, and the drug supplier user device 1008. The authentication block 1101 may be configured to enable the users' login by submission of authentication credentials. the patient user device 1002 may include a virtual pharmacy block 1102a, a prescription fulfillment block 1102b, a prescription auction block 1102c, a collaboration block 1102d, a prescription refill alerts block 1102e, a product offer alerts block 1102f, a doctor and pharmacist chats block 1102g, the personal wise health assistant module 207, a drug related analysis block 1102h, a marketplace block 1102i and a records of prescription block 1102j, a pharmacy preferences block 1102k, a prescription auto-renewal block 11021, and pharmacy locations 1102m, a medication intake schedule/alerts block 1102n, a fulfillment/shipment alerts block 1102o. The virtual pharmacy block 1102a may be configured to provide the virtual pharmacy configured to enable the patients to purchase medicine and drug/supplements based on their prescriptions. The virtual pharmacy may be an online representation of a pharmacy. The prescription fulfillment block 1102b may be communicated with the records of prescriptions block 1102j to display the prescriptions are currently in use, what prescriptions are completed, pending auto-renewals where there is need to have access, and to visualize. The prescription auction block 1102c may be configured to enable the patients to post the prescriptions for auction. The collaboration block 1102d may be communicated with the prescription refill alerts block 1102e, the product offer alerts block 1102f, the doctor and pharmacist chats block 1102g to generate the alerts to the patient. The alerts may include, but not limited to, prescription refill alerts, product offer alerts, doctor and pharmacist chat alerts, and so forth. The personal wise health assistant module 207 may be configured to provide helpful tips that may improve the patient's daily health. The drug related analysis block 1102h may be configured to provide drug related information and analysis. The marketplace block 1102i may be configured to provide marketplace experiences to the patients.

The prescription fulfillment module 203 may include the pharmacy preferences block 1102k, the prescription auto-renewal block 11021, and pharmacy locations 1102m. The pharmacy preferences block 1102k may be configured to provide preferable pharmacies to the patients. The prescription auto-renewal block 11021 may be configured to generate prescription auto-renewal alerts. The pharmacy locations block 1102m may be configured to provide preferable pharmacies to the patients. The collaboration module 205 may include the prescription refill alerts block 1102e, the medication intake schedule/alerts block 1102n, the product offer alerts block 1102f, and the fulfillment/shipment alerts block 1102o. The prescription refill alerts block 1102e may be configured to generate refill alerts and the product offer alerts block 1102f may be configured to generate product offer alerts on the patient user device 1002. The medication intake schedule/alerts block 1102n may be configured to generate medication intake schedule alerts on the patient user device 1002. The fulfillment/shipment alerts block 1102o may be configured to generate fulfillment/shipment alerts on the patient user device 1002.

The patient user device 202 further includes the prescription fulfillment module 203, the collaboration module 205, the personal wise health assistant module 207, the drug related analytics module 211, the marketplace module 213, a prescription block 1103a, a product offers block 1103b, a supplement/natural and drug advertisement block 1103c, a daily health suggestions and personalized doctor recommendations block 1103d, a Minor Diagnosis block 1103e, and a supplement/natural and drug replacement suggestions block 1103f. The prescription block 1103a may be configured to display the prescriptions are currently in use, what prescriptions are completed, pending auto-renewals where there is a need to have access, and to visualize important prescription information. The product offers block 1103b may be configured to generate product offers on the patient user device 1002. The supplement/natural and drug advertisement block 1103c may be configured to provide supplement/natural and drug advertisements on the patient user device 1002. The daily health suggestions and personalized doctor recommendations block 1103d may be configured to generate daily health suggestions and personalized doctor recommendations on the patient user device 1002. The minor diagnosis block 1103e may be configured to give suggestions of minor diagnosis on the patient user device 1002. The supplement/natural and drug replacement suggestions block 1103f may be configured to provide supplement/natural and drug replacement suggestions.

The drug related analysis module 211 includes a detailed drug and supplement/natural medicine information block 1105a, a drug information interface 1105b, an AI image to text conversion block 1105c, a cross-border drug conversion block 1105d, a drug conflicts block 1105e, a patient allergies block 1105f. The detailed drug and supplement/natural medicine information block 1105a may be configured to provide detailed drug and supplement/natural medicine information including chemicals and side effects. The drug information interface 1105b may be an interface between the drug related analysis module 211 and the marketplace module 213. The AI image to text conversion block 1105c may be configured to convert the artificial intelligence image to text to the patient on the patient user device 1002. The cross-border drug conversion block 1105d may be configured to generate cross-border drug conversions. The drug conflicts block 1105e may be configured to provide drug conflicts and the patient allergies block 1105f configured to provide the patient allergies.

The virtual pharmacy and consultation module 217 includes the prescription block 1107a, a view videos based on prescriptions block 1107b, a purchase medicine based on prescription block 1107c, an AI recommendations based on previous purchases block 1107d. The prescription block 1107a may be configured to display the prescriptions are currently in use, what prescriptions are completed, pending auto-renewals where there is need to have access, and to visualize. The view videos based on prescriptions block 1107b may be configured to enable the patient to view prescription videos and the pharmacy locations 1102m may give the exact location of the one or more pharmacies. The pharmacy locations 1102m may be communicated with the purchase medicine based on prescription block 1107c configured to enable the patient to purchase medicine based on prescriptions. The AI recommendations based on previous purchases block 1107d may be configured to generate recommendations via the collaboration module 205 on the patient user device 1002 based on previous purchases.

The prescription auction module 211 includes a personal settings block 1109a, a prescription not visible to other patients block 1109b, an ability to post prescriptions to auction interface 1109c, the prescriptions block 1109d, a receive alerts and updates on posts block 1109e. The personal settings block 1109a may be configured to enable the patient to control which pharmacies, for example, pharmacy locations 1102m can view the auction post. The ability to post prescriptions to auction interface 1109c may be configured to allow the patient to post auction prescriptions. The prescriptions block 1109d may be configured to display the prescriptions are currently in use, what prescriptions are completed, pending auto-renewals where there is a need to have access, and to visualize. The receive alerts and updates on posts block 1109e may be configured to receive alerts and updates on auction posts via the collaboration module 205.

The doctor user device 1006 includes the virtual pharmacy block 1104a, the prescription fulfillment block 1104b, the prescription auction block 1104c, the collaboration block 1104d, an automatically sending to pharmacists block 1104e, patient information, history, and prescriptions block 1104f, a speech to text conversion block 1104g, a nearby pharmacy information block 1104h, the custom patient related reminders block 1104i, the patient and pharmacist chats block 1104j, the patients' service alerts block 1104k, the patient auto-renewal or refill request block 11041, the rate, approve and comment on videos and medicine block 1104m, and rate and approve certified vendors and recommended pharmacies block 1104n. The virtual pharmacy block 1104a may be configured to provide the virtual pharmacy configured to enable the doctors to purchase medicine and drug/supplements based on the received prescription. The prescription fulfillment block 1104b may be configured to communicate with the automatically sending to pharmacists block 1104e, and the patient information, history, and prescriptions block 1104f to generate prescription fulfillment alerts to pharmacists, and to get patient information, history, and prescriptions. The prescription fulfillment block 1104b may also communicate with the speech to text conversion block 1104g to convert the speech to text on the doctor user device 1006. The prescription fulfillment block 1104b may also communicate with the nearby pharmacy information block 1104h to generate nearby pharmacy information on the doctor user device 1006. The prescription auction block 1104c may be configured to generate auction alerts on the doctor user device 1006 via the collaboration block 1104d. The custom patient related reminders block 1104i may be configured to generate custom patient related reminders on the doctor user device 1006 via the collaboration block 1104d. The patient and pharmacist chats block 1104j may be configured to enable the doctor to chat with the patient and also the pharmacist via the collaboration block 1104d.

The patient service alerts block 1104k may be configured to generate patient service alerts on the doctor user device 1006 via the collaboration module 205. The patient auto-renewal or refill request block 11041 may be configured to generate patient auto-renewal or refill requests on the doctor user device 1006 via the collaboration module 205. The virtual pharmacy and consultation module 217 includes the rate, approve and comment on videos and medicine block 1104m configured to give rate, approval, and comment on videos and medicine. The prescription auction module 219 includes a rate and approve certified vendors and recommended pharmacies block 1104n configured to give rate and approve certified vendors and recommended pharmacies.

The pharmacist user device 1004 includes the virtual pharmacy block 1106a, the prescription fulfillment/shipment block 1106b, the prescription auction block 1106c, a pending prescription log block 1106d, the collaboration block 1106e, an upcoming and pending patient orders block 1106f, a patient and doctor chats block 1106g, a pharmacy system integration block 1106h, a patient auto-renewal or refill request block 1106i, an upcoming and pending patient orders block 1106f, an ability to post instructional videos block 1106j, a post medicine for purchase 1106k, an ability to place bids against other pharmacies block 11061, fulfil the prescription if they win the auction bid 1106m, ability to filter postings based on price, location, stock 1106n, and receive alerts and updates based on auctions they follow 1106o. The virtual pharmacy block 1106a may be configured to provide the virtual pharmacy configured to enable the pharmacist to sell medicine and drug/supplements based on the received prescription. The prescription fulfillment/shipment block 1106b may be configured to communicate with the pending prescription log block 1106d to provide pending prescription logs on the pharmacist user device 1004. The prescription auction block 1106c may be configured to enable the pharmacist to post the prescriptions. The upcoming and pending patient orders block 1106f may be communicated with the collaboration block 1106e configured to generate upcoming and pending patient orders. The patient and doctor chats block 1106g may be configured to enable the pharmacist to chat with patient and doctor in real-time. The prescription fulfillment module 203 includes the prescription fulfillment/shipment block 1106b, and the pending prescription log block 1106d.

The pharmacy system integration block 1106h may be configured to integrate with the doctor system integration block to communicate the pending prescription logs, and the prescription fulfillment/shipment. The upcoming and pending patient orders block 1106f may be configured to generate upcoming and pending patient orders on the pharmacist user device 1004 via the collaboration module 205. The patient auto-renewal or refill request block 1106i may be configured to generate auto-renewal or refill requests on the pharmacist user device 1004 via the collaboration module 205. The virtual pharmacy and consultation module 217 includes the ability to post instructional videos block 1106j configured to enable the pharmacist to post instructional videos. The post medicine block 1106k may be configured to enable the pharmacist to post medicine for purchase.

The prescription auction module 219 includes the ability to place bids against other pharmacies block 11061, fulfil the prescription if they win the auction bid 1106m, the ability to filter postings based on price, location, stock 1106n, and the receive alerts and updates based on auctions they follow 1106o. The ability to place bids against other pharmacies block 11061 may be configured to enable the pharmacist to place bids against other pharmacies. Fulfil the prescription if they win the auction bid 1106m may be configured to enable the pharmacist to fulfil the prescription if the pharmacist wins the auction bid. The ability to filter postings based on price, location, stock 1106n may be configured to enable the pharmacist to filter postings based on price, location, and stock. The receive alerts and updates based on auctions they follow 1106o may be configured to enable the pharmacist to receive alerts and updates based on auctions they follow.

The drug supplier user device 1008 includes a prescription auction block 1108a, a marketplace block 1108b, a posting ads for health suggestions block 1108c, a consumer traffic on selected products block 1108d, a custom product postings block 1108e, a Drug, Suppliers/Natural Medicine Providers block 1108f, an offer bundles of products on prescription postings block 1108g, AI Recommendations based on previous user purchases block 1108h. The prescription auction block 1108a may be configured to enable the drug supplier to post the prescriptions. The marketplace block 1108b may be communicated with the posting ads for health suggestions block 1108c, the consumer traffic on selected products block 1108d, and the custom product postings block 1108e. The posting ads for health suggestions block 1108c may be configured to enable the drug supplier to post ads for health suggestions. The consumer traffic on selected products block 1108d may be configured to enable the drug supplier to view consumer traffic on selected products. The custom product postings block 1108e may be configured to enable the drug supplier to post custom products. The Drug, Suppliers/Natural Medicine Providers block 1108f may be communicated with the marketplace module 213. The Drug, Suppliers/Natural Medicine Providers block 1108f may include a list of drug suppliers/natural medicine providers. They offer bundles of products on prescription postings block 1108g may be configured to provide bundles of products on prescription postings. The AI Recommendations based on previous user purchases block 1108h may be configured to generate recommendations based on previous user purchases.

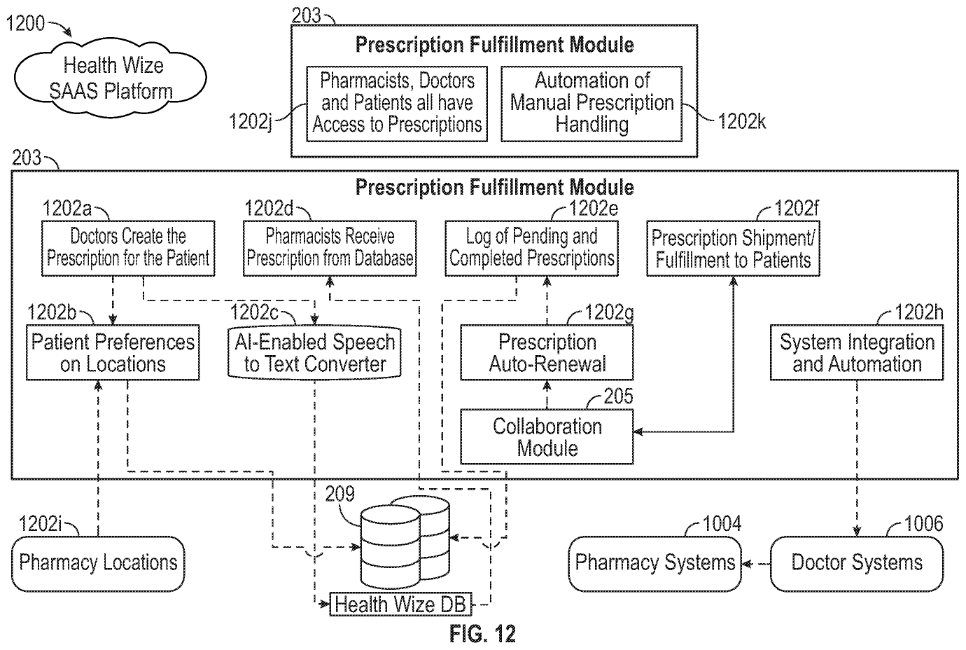

Referring to FIG. 12 is an example block diagram 1200 depicting the prescription fulfillment module 203, in accordance with one or more exemplary embodiments. The prescription fulfillment module 203 includes a create prescription block 1202a, a patient preferences block 1202b, a speech to text converter 1202c, a receive prescription block 1202d, a prescription log 1202e, a prescription shipment/fulfillment block 1202f, the collaboration module 205, a prescription auto renewal block 1202g, the system integration and automation block 1202h, pharmacy locations 1202i, a Prescriptions access block 1202j, a prescription handling block 1202k, the database 209, the pharmacist system 1004 and the doctor system 1006. The create prescription block 1202a may be configured to enable the doctors to create the prescriptions for the patient. The patient preferences block 1202b may be communicated with the pharmacy locations 1202i configured to provide preferable pharmacy locations. The speech to text converter 1202c may be configured to convert the speech to text through an artificial intelligence technique. The receive prescription block 1202d may be configured to enable the pharmacists to receive prescriptions.

The prescription log 1202e may be configured to generate a log of pending prescriptions and completed prescriptions from the database 209. The prescription shipment/fulfillment block 1202f may also communicate with the collaboration module 205 to generate prescription information on the patient user device 1002/the pharmacist user device 1004/doctor user device 1006. The prescription auto renewal block 1202g may be configured to generate prescription auto renewals via the collaboration module 205. The system integration and automation block 1202h may be configured to communicate with the pharmacist system 1004 and the doctor system 1006.

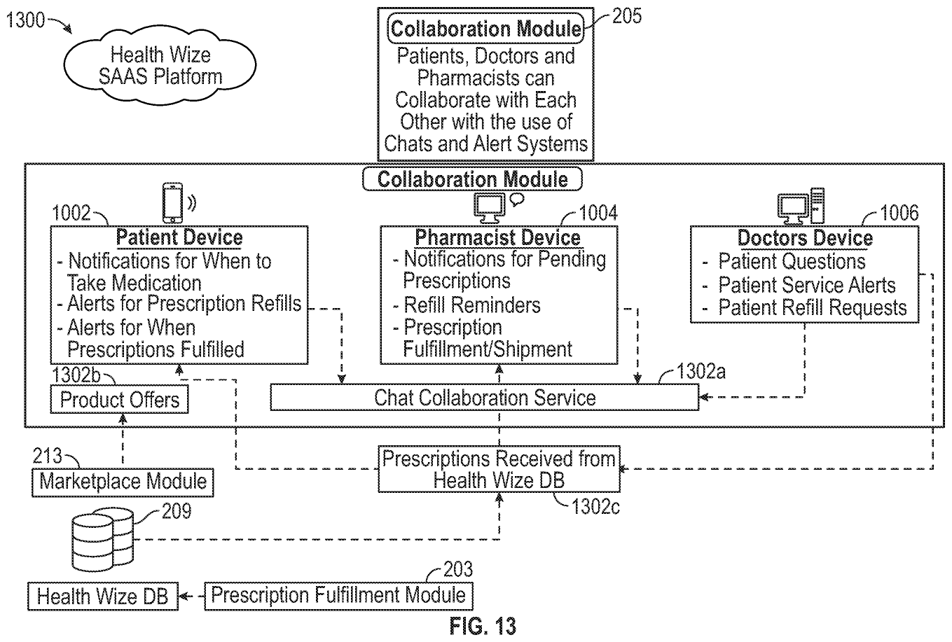

Referring to FIG. 13 is an example block diagram 1300 depicting the collaboration module 205, in accordance with one or more exemplary embodiments. The collaboration module 205 includes a chat collaboration service block 1302a, a product offers block 1302b, a prescription receive block 1302c, the patient user device 1002, a pharmacist user device 1004, the doctor user device 1006, and the database 209. The chat collaboration service block 1302a may be configured to establish communication between the patient user device 1002, the pharmacist user device 1004, and the doctor user device 1006. The patient user device 1002 may be configured to generate notifications for when to take medication, alerts for prescription refills, and alerts for when prescriptions fulfilled. The pharmacist user device 1004 may be configured to generate notifications for pending prescriptions, refill reminders, prescription fulfillment/shipment. The doctor user device 1006 may be configured to generate patient questions, patient service alerts, and patient refill requests. The product offers block 1302b may be communicated with the marketplace module 213 configured to generate product offers to the patient and the pharmacist. The prescription receive block 1302c may be communicated with the database 209 configured to receive prescriptions. The prescription fulfillment module 203 may be communicated with the database 209.

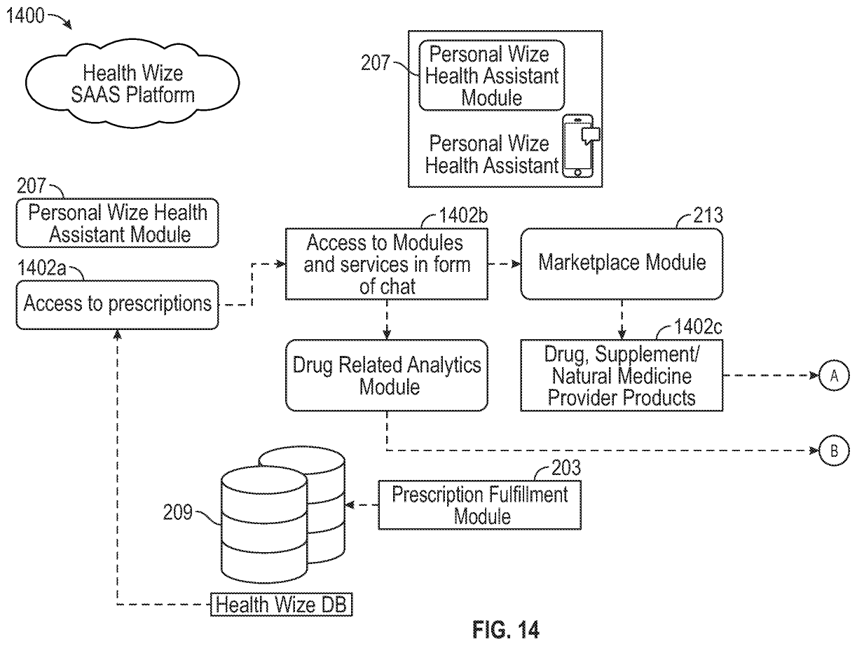

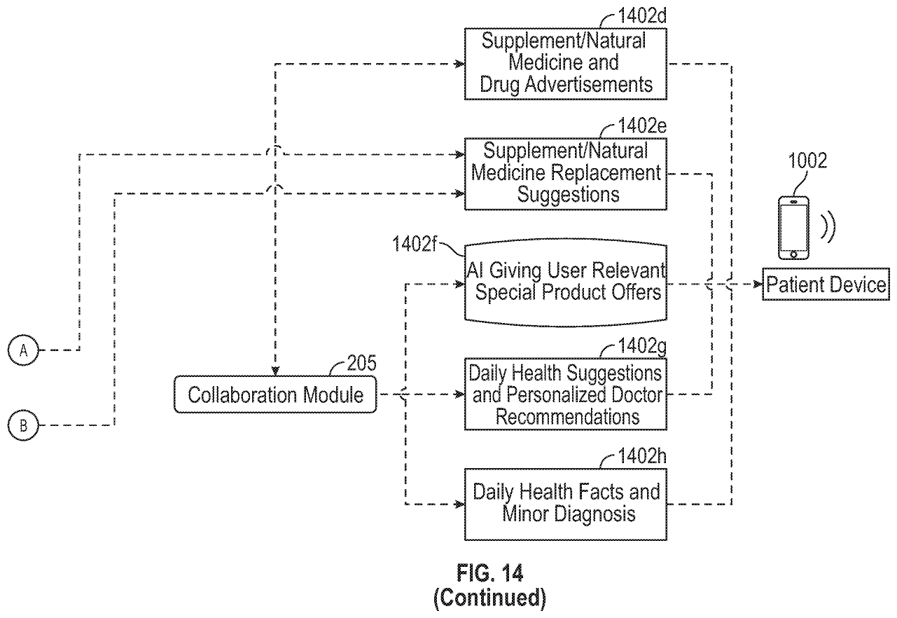

Referring to FIG. 14 is an example block diagram 1400 depicting the health assistant module 207, in accordance with one or more exemplary embodiments. The health assistant module 207 includes the prescriptions block 1402a, a chat block 1402b, a products block 1402c, an advertisement block 1402d, a suggestion block 1402e, an offers block 1402f, a recommendation block 1402g, and a health facts and minor diagnosis block 1402h. The prescriptions block 1402a may be configured to enable the user to access the prescriptions received from the database 209. The chat block 1402b may be configured to enable the users to access the modules and services in the form of chat. The products block 1402c may be configured to provide a list of drugs, supplement/natural medicine on the computing device. The advertisement block 1402d may be configured to generate supplement/natural medicine and drug advertisements. The suggestion block 1402e may be configured to generate supplement/natural medicine replacement suggestions. The offers block 1402f may be configured to give user Relevant Special Product Offers using the artificial intelligence technique. The recommendation block 1402g may be configured to generate daily health suggestions and personalized doctor recommendations via the collaboration module 205. The health facts and minor diagnosis block 1402h may be configured to provide daily health facts and minor diagnosis via the collaboration module 205.

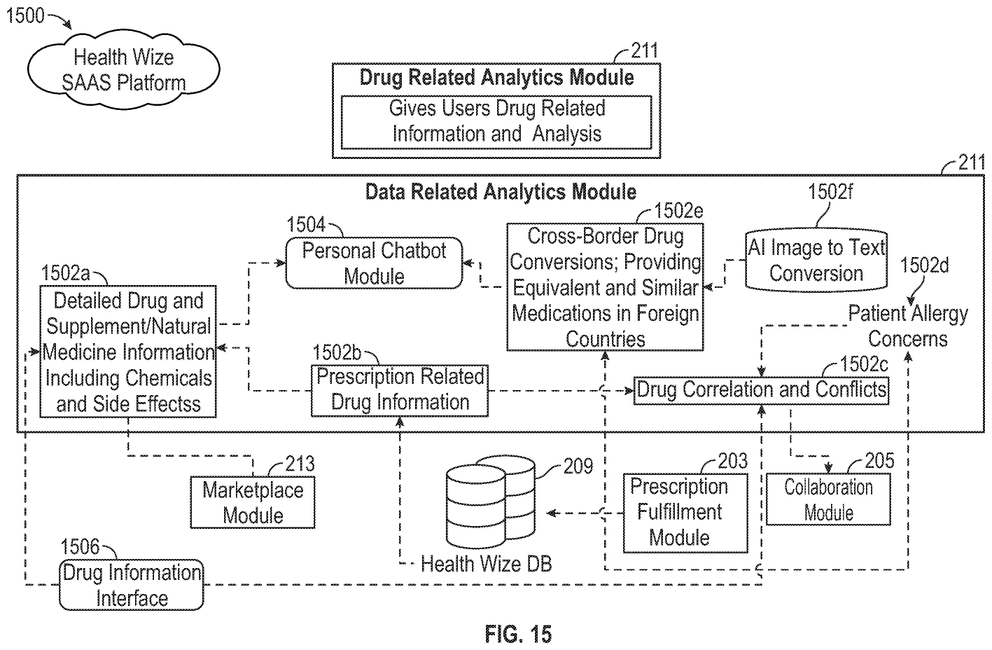

Referring to FIG. 15 is an example block diagram 1500 depicting the drug related analytics module 211, in accordance with one or more exemplary embodiments. the drug related analytics module 211 includes a chemicals and side effects block 1502a, a personal chatbot module 1504, a prescription related drug information block 1502b, a drug correlation and conflicts block 1502c, a patient allergy concerns block 1502d, the cross-border drug conversions block 1502e, the image to text conversion block 1502f, the database 209, and a drug information interface 1506.

The chemicals and side effects block 1502a may be configured to provide detailed drug and supplement/natural medicine information through the personal chatbot module 1504. The medicine information includes chemical information, side effects information, and so forth. The prescription related drug information block 1502b may be configured to collect prescription related drug information from the database 209. Where the prescription fulfillment module 203 may send prescription information to the database 209. The prescription related drug information block 1502b may be communicated with the chemicals and side effects block 1502a and the drug correlation and conflicts block 1502c. The drug correlation and conflicts block 1502c may be configured to correlate and conflict the medical information and transmit the alerts through the collaboration module 205. The patient allergy concerns block 1502d may be configured to provide patient allergy concerns. The cross-border drug conversions block 1502e may be configured to provide the cross-border drug conversions and also equivalent and similar medications in foreign countries through the personal chatbot module 1504. The image to text conversion block 1502f may be configured to convert the images to text using an artificial intelligence technique. The converted text may include but not limited to, cross-border drug conversions, equivalent and similar medications in foreign countries, and so forth. The drug information interface 1506 may be an interface between the chemicals and side effects block 1502a and the drug correlation and conflicts block 1502c.

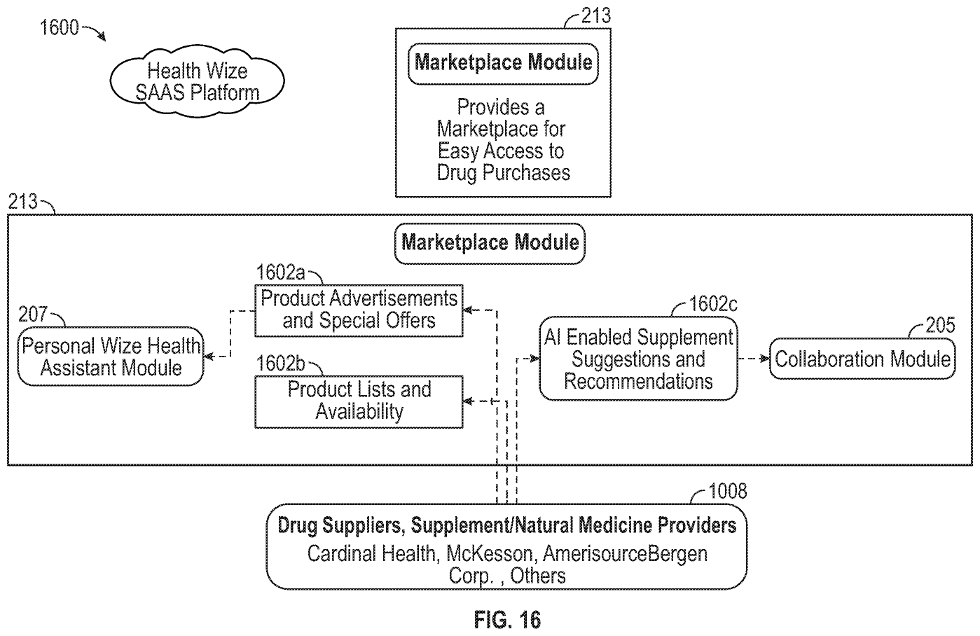

Referring to FIG. 16 is an example block diagram 1600 depicting the marketplace module 213, in accordance with one or more exemplary embodiments. The marketplace module 213 includes an advertisement and offers block 1602a, a product list and availability block 1602b, and an AI enabled supplement suggestions and recommendations block 1602c. The advertisement and offers block 1602a may be configured to enable the fourth user and the fifth user on the drug supplier user device 1008 to generate advertisements and offers to the first user and the second user through the health assistant module 207. The product list and availability block 1602b may be configured to enable the fourth user and the fifth user on the drug supplier user device 1008 to provide the list of available products. The list of available products may include but not limited to, medicines, medical devices, drugs, and so forth. The AI enabled supplement suggestions and recommendations block 1602c may be configured to enable the fourth user and the fifth user on the drug supplier user device 1008 to provide supplement suggestions and recommendations. The supplement suggestions and recommendations may be transmitted to the first user through the collaboration module 205.

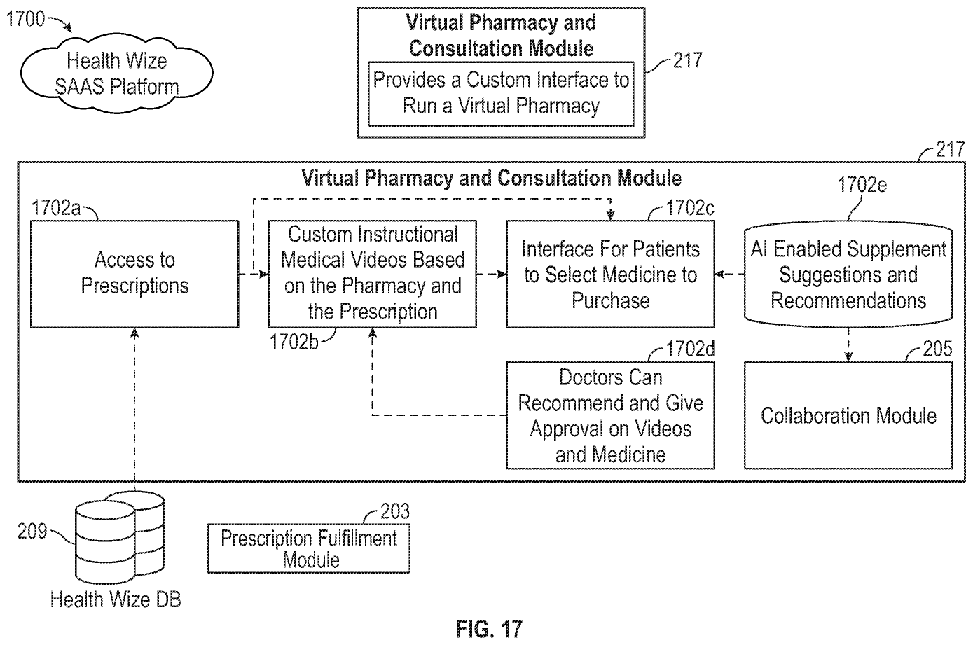

Referring to FIG. 17 is an example block diagram 1700 depicting the virtual pharmacy and consultation module 217, in accordance with one or more exemplary embodiments. The virtual pharmacy and consultation module 217 includes the prescription block 1702a, a custom instructional medical videos block 1702b, an interface block 1702c, a recommendation block 1702d, an AI enabled supplement suggestions and recommendations block 1702e. The prescription block 1702a may be configured to enable the first user to access the prescriptions received from the database 209 and also configured to enable the first user to select the medicine to purchase via the interface block 1702c. The interface block 1702 may be the interface for the first user to select medicine to purchase. The custom instructional medical videos block 1702b may be configured to provide custom instructional medical videos based on the pharmacy and the prescription. The recommendation block 1702d may be configured to recommend videos and also enables the second user to approve the videos on the doctor user device 1004. The AI enabled supplement suggestions and recommendations block 1702e may be configured to enable the fourth user and the fifth user to provide supplement suggestions and recommendations using the artificial intelligence technique.

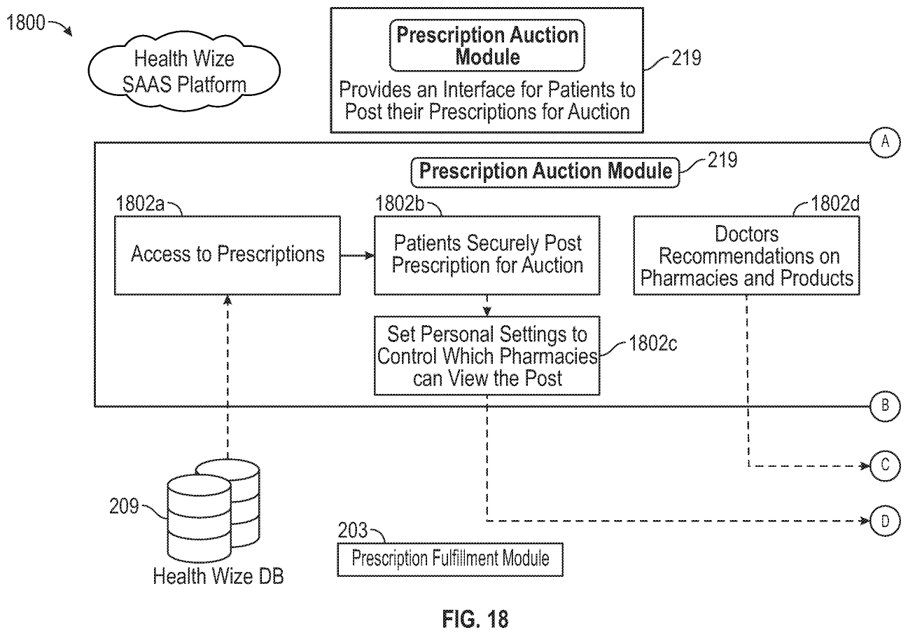

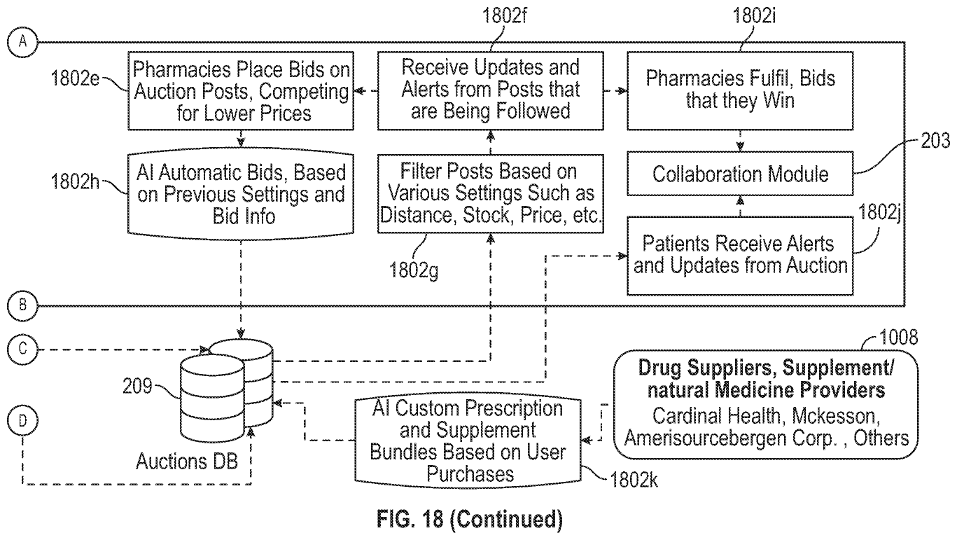

Referring to FIG. 18 is an example block diagram 1800 depicting the prescription auction module 219, in accordance with one or more exemplary embodiments. The prescription auction module 219 includes the prescription block 1802a, a post prescription block 1802b, a personal settings block 1802c, a recommendations block 1802d, a bids on auction posts block 1802e, a pharmacies' updates and alerts block 1802f, a filter posts block 1802g, an AI automatic bids block 1802h, a bids block 1802i, a Patients' alerts and updates block 1802j, and an AI custom prescription and supplement bundles block 1802k.

The prescription block 1802a may be configured to enable the first user to access the prescriptions received from the database 209 and also configured to enable the first user to post the prescription for auction. The personal settings block 1802c may be configured to enable the first user to control which pharmacies can view the auction post. The recommendations block 1802d may be configured to enable the second user to provide recommendations on pharmacies and products. The bids on auction posts block 1802e may be configured to enable the third users, for example, pharmacies to place bids on auction posts, competing for lower prices. The AI automatic bids block 1802h may be configured to transmit placed bids using the artificial intelligence technique to the database 209 based on previous settings and bid information. The filter posts block 1802g may be configured to enable the third user to filter posts based on various settings such as distance, inventory and stock, services, price, and so forth. The pharmacies' updates and alerts block 1802f may be configured to enable the third users to receive updates and alerts from posts that are being followed. The bids block 1802i may be configured to provide how many bids that the third user wins via the collaboration module 205 on the pharmacist user device 1006. The Patients' alerts and updates block 1802j may be configured to enable the first user to receive alerts and updates from the auction database 209. The drug supplier user device 1008 may be configured to generate custom prescription and supplement bundles via the AI custom prescription and supplement bundles block 1802k and then send it to the database 209.

According to exemplary embodiments of the present disclosure, the artificial intelligent platform 100 for collaborating, automating and organizing health-information between users in the pharmaceutical/healthcare industries as well as users in the veterinarian space. The artificial intelligent platform 100 includes a medical information generation module 114 configured to eliminate the tedious hard copy exchange and execution of a plurality of prescriptions by automating communication and fulfillment between a plurality of computing devices 102/104/106/108/110/1002/1004/1006/1008/1010. The plurality of computing devices 102/104/106/108/110/1002/1004/1006/1008/1010 may be configured to enable a plurality of users to access the plurality of prescriptions on the medical information generation module 114, the medical information generation module 114 comprises a prescription fulfillment module 203 configured to cover all steps of a typical prescription uses the medical information collected from a database 209 to display different information on the plurality of computing devices 102/104/106/108/110/1002/1004/1006/1008/1010 based on the plurality of users regarding the fulfillment of the prescription, the prescription fulfillment module 203 also comprises the automation of prescription handling by integrating with a plurality of healthcare/pharmacy/veterinary systems to provide a secure and streamlined communication system.

According to exemplary embodiments of the present disclosure, the artificial intelligent platform 100 comprises the collaboration module 205 may be configured to collaborate the plurality of users with each other comprising of a direct communication chat function and also configured to generate a plurality of real time alerts to proper users on the plurality of computing devices for a plurality of reminders such as prescription refill reminders and updates, key health check recommendation reminders, and due lab work reminders. The health assistant module 207 may be configured to provide a plurality of customized suggestions and recommendations from artificial intelligence models on the plurality of computing devices. The plurality of customized suggestions and recommendations may include, but not limited to, supplements, services, consultations, and so forth. The health assistant module 207 may be configured to communicate with the collaboration module 205 and the marketplace module 213 to display prescription information, stakeholder information, patient information and history, a plurality of products and a plurality of offers on the plurality of computing devices. The stakeholder information may include, clinic addresses, frequently visited doctors, recommended pharmacies, and so forth.

According to exemplary embodiments of the present disclosure, the artificial intelligent platform 100 includes the analytics module 211 configured to convert a plurality of images to text using artificial intelligence techniques on the plurality of computing devices 102/104/106/108/110/1002/1004/1006/1008/1010, the text is used to capture drug information and correlations, and cross border drug conversions. The artificial intelligent platform 100 includes the marketplace module 213 configured to provide easy access for the plurality of users to view, purchase and receive the plurality of items such as drugs, supplements, vitamins, hygienic products, Ayurveda, health related services, yoga, consultations, and life coaches services. The marketplace module 213 may be configured to allow a plurality of drug manufacturers, supplements providers and service providers to display, sell and control products they offer using artificial intelligence, product and services recommendations based on past prescriptions and purchases.

According to exemplary embodiments of the present disclosure, the artificial intelligent platform 100 includes a virtual pharmacy and consultation module 217 configured to provide a virtual pharmacy that enables the plurality of users on the plurality of computing devices 102/104/106/108/110/1002/1004/1006/1008/1010 to purchase medicine and drug/supplements virtually based on the plurality of prescriptions as well as view videos and have video consultations with the plurality of users. The prescription auction module 219 may be configured to allow a plurality of first users to upload the plurality of prescriptions and consultation services to an auction post on the plurality of computing devices 102/104/106/108/110/1002/1004/1006/1008/1010, a plurality of second users able to view and bid the auction post on the plurality of computing devices 102/104/106/108/110/1002/1004/1006/1008/1010 and then fulfil the plurality of prescriptions or required services after winning the auction.

According to exemplary embodiments of the present disclosure, the prescription fulfillment module 203 may be configured to convert prescription data from an image and using artificial intelligence detect/understand the information within a typical patient/veterinarian prescription, including patient details, drug name and amounts, date and time of assigned prescription, and drug intake instructions. The prescription fulfillment module 203 may be configured to integrate with the plurality of healthcare/pharmacy/hospital/medical/veterinarian systems. The plurality of healthcare/pharmacy/hospital/medical/veterinarian systems may include, but not limited to, pharmacy management systems (PMS), electronic medical records (EMR), hospital management systems, veterinary management systems, and so forth. The integration includes but not limited to the automation of data filing from prescriptions and other sources of stored data as well as inter-system communication to manage a plurality of local functionality from the cloud server 215. The collaboration module 205 may be configured to provide a common cloud platform in which the plurality of first users may directly communicate and collaborate with the plurality of second users and a plurality of third users. Communication and collaboration are in the forms of direct chat messaging, video conferencing, internet calling, and so forth. The collaboration module 205 may be configured to allow the easy collaboration of a plurality of services by providing an easy way to execute the plurality of services and functionality provided by the platform including, prescription corrections, prescription refills, supplement recommendations, and links to specific auction posts.

According to exemplary embodiments of the present disclosure, the collaboration module 205 may be configured to send the plurality of real time alerts between the plurality of first users, the plurality of second users, the plurality of third users, a plurality of fourth users, and a plurality of fifth users. The alerts may be configured and customized to the specific functionality and needs that the healthcare/pharmacy/veterinary clinics require. Different alerts exist depending on the user. Possible alerts and customizations include, but not limited to, refill reminders, prescription fulfillment or shipment, product offers based on artificial intelligence models, pending prescriptions, and so forth.

According to exemplary embodiments of the present disclosure, the analytics module 211 may be configured to send the plurality of first users with information and insights on a plurality of drugs. There are a plurality of ways to receive the information and insights from the artificial intelligent platform 100. The plurality of users may reference drugs/supplements/vitamins that are included on their prescription, take pictures of drugs/supplements/vitamins/Ayurveda and use artificial intelligence to detect and understand the text in the picture, reference products listed in the marketplace. When information about a product is detected, there are a plurality of insights and information to give. These include, drug information (for example name, chemicals, side effects, etc.), cross-border drug conversions (name-brand drugs that contain the same chemical but have different brand names depending on the country), drug correlations and conflicts, reviews based on the marketplace reviews for the specific product, and so forth.

According to exemplary embodiments of the present disclosure, the marketplace module 213 may be configured to host a safe and secure marketplace for the plurality of users to purchase and list drugs, supplements, natural medicine. Users have the plurality of capabilities including viewing product information, purchasing products, sending reviews on products, and so forth. The plurality of fourth users and fifth users (drug, supplement, consultations and service providers) have a plurality of configurations that include hosting products based on inventory, monitor postings, apply deals sales and discounts, dispatch advertisements.

According to exemplary embodiments of the present disclosure, the virtual pharmacy and consultation module 217 may be configured to allow the plurality of second users (pharmacists) to host pharmacies virtually. The virtual pharmacy and consultation module 217 may be configured to may be configured to allow pharmacists to configure which drugs, supplements, services etc. are available for purchase via the user interface. Pharmacies may also integrate their local stock and inventory system to handle inventory management through the interface. This allows a plurality of first users to upload their prescription for approval and fulfil the prescription from anywhere.

According to exemplary embodiments of the present disclosure, the virtual pharmacy and consultation module 217 may be configured to have an interface to host custom instructional videos based on the plurality of prescriptions. These videos may be tailored to the prescriptions that are given to the plurality of first users. The instructional videos may include but are not limited to, instructions for medicine intake/application, instructional videos for physiotherapy, and so forth. These videos may be accessed by a plurality of users, and can also receive ratings by a plurality of third users (doctors).

According to exemplary embodiments of the present disclosure, the virtual pharmacy and consultation module 217 may be configured to have a function for a plurality of second users to host their practice virtually through video consultation. The second users such as doctors, pharmacists, veterinarians, physiotherapists, psychiatrists, life coaches, and so forth. The virtual pharmacy and consultation module 217 may have all functionality that would help the second users their practice virtually including, scheduling meetings, receiving payments, assigning tasks, assigning prescriptions, and so forth.

According to exemplary embodiments of the present disclosure, the prescription auction module may be configured to allow the plurality of first users to post prescriptions and services up for bidding/auction. Once the post is up for a bid/auction, then the plurality of users such as doctors, pharmacists, veterinarians, physiotherapists, psychiatrists, life coaches, and so forth, may bid on the post. Once an auction is over, the user with the best price and service may then fulfill the prescription or service required. There are multiple filters that a plurality of users may modify to view the auctions including distance, service, price, stock and inventory, delivery availability and so forth. The users may also view a log of completed auctions.

According to exemplary embodiments of the present disclosure, the health assistant module 207 may be configured to provide suggestions from artificial intelligence models and also configured to other modules to give the user easy access to trigger services. A plurality of users can access a chatbot (built on AI) that will understand questions asked and respond accordingly or execute a particular service through the platform. The chatbot can also give a plurality of recommendations based on patient information and history. Using artificial intelligence, the chatbot may give suggestions including supplements that go well with medication on the prescription, suggestions to mitigate medicine side effects, service or exercise suggestions based on chronic conditions, and so forth. The health assistant module 207 may also access the other modules (prescription fulfillment module 203, the Collaboration Module 205, prescription auction module 219, marketplace module 213) to give the user easy access to trigger services throughout the platform.

According to exemplary embodiments of the present disclosure, all modules extend and may be modified for the veterinary industry. For example the plurality of users may use the prescription fulfillment module 203 to fulfill prescriptions for animals, users may use the collaboration module 205 to directly communicate for help with their animals, users may use the analytics module 211 to get more information on the drugs or supplements their pets are taking, users can use the marketplace module 213 to have safe and secure access to animal drugs/supplements, users may use the virtual pharmacy and consultation module 217 to receive virtual consultations and diagnosis for their animals, users may use the prescription auction module 219 to post prescriptions or services on the auction interface for their pet, users can use the health assistant module 207, to ask a chatbot for advice and receive suggestions for their animal, and so forth.

According to exemplary embodiments of the present disclosure, the prescription fulfillment module 203 may be configured to display a plurality of pending prescriptions, prescriptions are currently in use, what prescriptions are completed, pending auto-renewals, and prescription progress where there is need to have an access and to visualize on the plurality of computing devices 102/104/106/108/110/1002/1004/1006/1008/1010. The plurality of users get constant and live updates as the prescription is getting completed to see which stage the prescription fulfillment is at. The prescription fulfillment module 203 may be also configured to display the plurality of first users to the plurality of second users that they are dealing with along with each user's current and completed prescriptions on the plurality of computing devices 102/104/106/108/110/1002/1004/1006/1008/1010. Patient's information may be easily accessible as they are linked to their prescription history. New patients are easily added by integration with pharmacy management systems (PMS), to add existing patient data to the artificial intelligent platform.

According to exemplary embodiments of the present disclosure, the wherein the health assistant module 207 comprises a correlation and conflict module 606 configured to correlate and conflict the medical information in the plurality of prescriptions and transmit the plurality of real time alerts through the collaboration module 205. When the plurality of users are using the health assistant, it can notify with real-time updates on prescriptions currently getting fulfilled. It may also alert if a certain drug on the marketplace conflicts with medicine on the prescription.

According to exemplary embodiments of the present disclosure, the medical information generation module 114 is configured to provide medical information such as past medical history, family medical history, medication history, personal and lifestyle history, psychological analysis, vital signs. electrocardiogram (EKG or ECGs), and other lab reports such as drug level, bacteria in urine, bacteria in the blood, activities of the individual, and glucose levels. These metrics are may be also extended to animals for the veterinary industry. The information may include the aforementioned metrics, along with animal specific metrics like animal specific diseases, castrations, breed, history, and so forth.

A method for collaborating and organizing medical information between multiple users in the pharmaceutical/healthcare industries, comprising: enabling a plurality of users to login to a medical information generation module 114 by creating user accounts using login identity credentials through a plurality of computing devices 102/104/106/108/110/1002/1004/1006/1008/1010, the medical information generation module 114 may be configured to provide a plurality of custom webpages based on the type of a user and execution of a plurality of prescriptions by automating communication and fulfillment between the plurality of computing devices 102/104/106/108/110/1002/1004/1006/1008/1010, enabling the plurality of users to access a plurality of medical prescriptions on the medical information generation module 114, the plurality of medical prescriptions are securely stored in a database 209, collaborating the plurality of users with each other and also generating a plurality of real time alerts to proper users on the plurality of computing devices 102/104/106/108/110/1002/1004/1006/1008/1010 for prescription refill reminders, key health check recommendation reminders, and due lab work reminders, generating medical related suggestions and recommendations using artificial intelligence on the plurality of computing devices 102/104/106/108/110/1002/1004/1006/1008/1010 by the medical information generation module 114, and providing a marketplace to a plurality of first users, a plurality of second users, and a plurality of third users on the plurality of computing devices 102/104/106/108/110/1002/1004/1006/1008/1010 through the medical information generation module 114 and an easy access to product purchases from a plurality of fourth users and a plurality of fifth users.

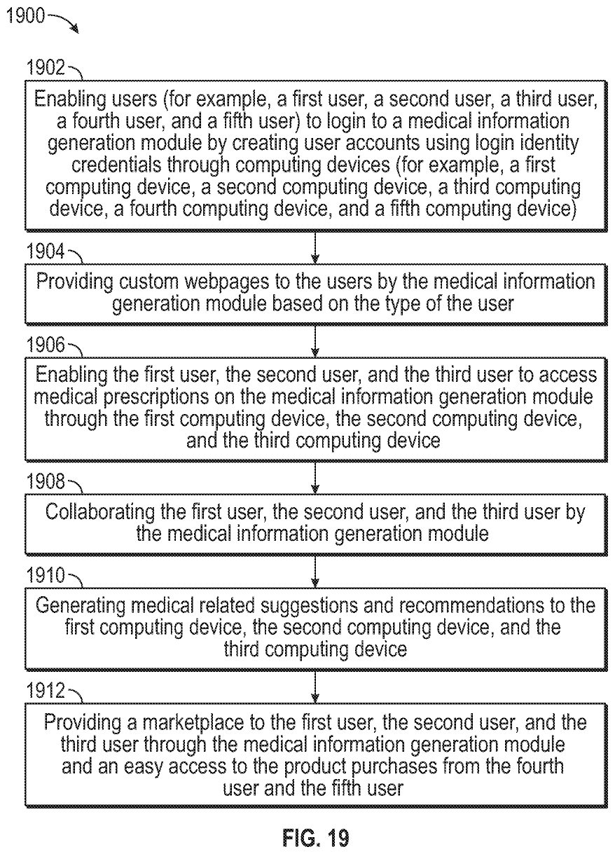

Referring to FIG. 19 is a flow chart 1900, depicting an exemplary method for collaborating and organizing medical information between multiple users in the pharmaceutical/healthcare industries, in accordance with one or more exemplary embodiments. As an option, the method 1900 may be carried out in the context of the details of FIG. 1, FIG. 2, FIG. 3, FIG. 4, FIG. 5, FIG. 6, FIG. 7, FIG. 8, FIG. 9, FIG. 10, FIG. 11, FIG. 12, FIG. 13, FIG. 14, FIG. 15, FIG. 16, FIG. 17, and FIG. 18. However, the method 1900 may also be carried out in any desired environment. Further, the aforementioned definitions may equally apply to the description below.

The method commences at step 1902, the users (for example, a first user, a second user, a third user, a fourth user, and a fifth user) are enabled to login the medical information generation module by creating user accounts using login identity credentials through computing devices (for example, a first computing device, a second computing device, a third computing device, a fourth computing device, and a fifth computing device). Here, the medical information generation module may determine the type of the user based on the login identity credentials. The type of the user may include but not limited to, doctor, patient, pharmacist, drug supplier, supplement provider, and so forth. The identity credentials may include but not limited to, a unique identifier (e.g., a username, an email address, a mobile number, a subscription identity, and so forth), and a secured code (e.g., a password, a symmetric encryption key, biometric values, a passphrase, and so forth). Thereafter, at step 1904, the medical information generation module provides the custom webpages based on the type of the user. Here, the webpages may include different service site webpages and modules which are relevant to the user. Thereafter, at step 1906, the medical information generation module enables the first user, the second user, and the third user to access medical prescriptions through the first computing device, the second computing device, and the third computing device. The medical prescriptions may be stored in the database/and or cloud server. Thereafter, at step 1908, the medical information generation module collaborates with the first user, the second user, and the third user through the chatbot. Thereafter, at step 1910, the medical information generation module generates medical related suggestions and recommendations to the first computing device, the second computing device, and the third computing device. Thereafter, at step 1912, the medical information generation module provides a marketplace to the first user, the second user, and the third user through the medical information generation module and an easy access to the product purchases from the fourth user and the fifth user.

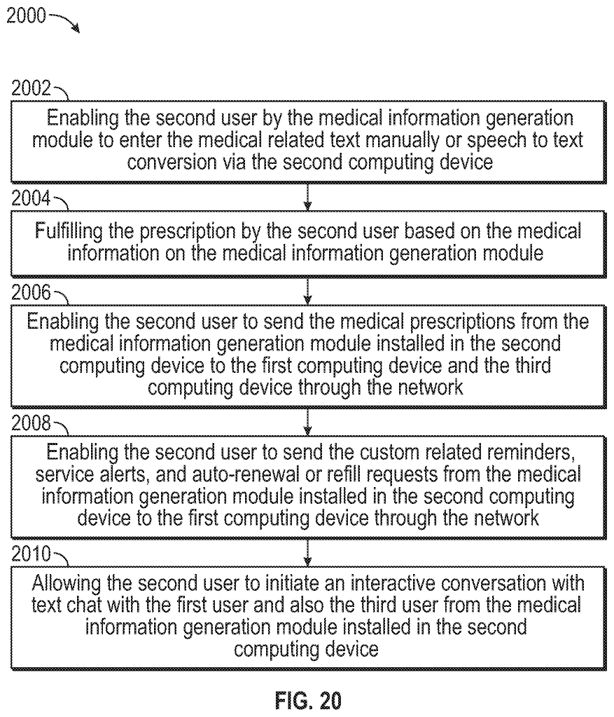

Referring to FIG. 20 is a flow chart 2000, depicting an exemplary method for collaborating the second user with the first user and the third user, in accordance with one or more exemplary embodiments. As an option, the method 1000 may be carried out in the context of the details of FIG. 1, FIG. 2, FIG. 3, FIG. 4, FIG. 5, FIG. 6, FIG. 7, FIG. 8, FIG. 9, FIG. 10, FIG. 11, FIG. 12, FIG. 13, FIG. 14, FIG. 15, FIG. 16, FIG. 17, FIG. 18, and FIG. 19. However, the method 2000 may also be carried out in any desired environment. Further, the aforementioned definitions may equally apply to the description below.

The medical information generation module enables the second user to enter the medical related text manually or speech to text conversion via the second computing device, at step 2002. Here, the medical information may include but not limited to, patient information, history, and previous prescriptions, and so forth. Thereafter, at step 2004, fulfilling the prescription by the second user based on the medical information on the medical information generation module. Thereafter, at step 2006, the medical information generation module enables the second user to send the prescriptions from the medical information generation module installed in the second computing device to the first computing device and the third computing device through the network. Thereafter, at step 2008, the medical information generation module enables the second user to send the custom related reminders, service alerts, and auto-renewal or refill requests from the medical information generation module installed in the second computing device to the first computing device through the network. Thereafter, at step 2010, the medical information generation module allows the second user to initiate an interactive conversation with the first user and also the third user from the medical information generation module installed in the first computing device.

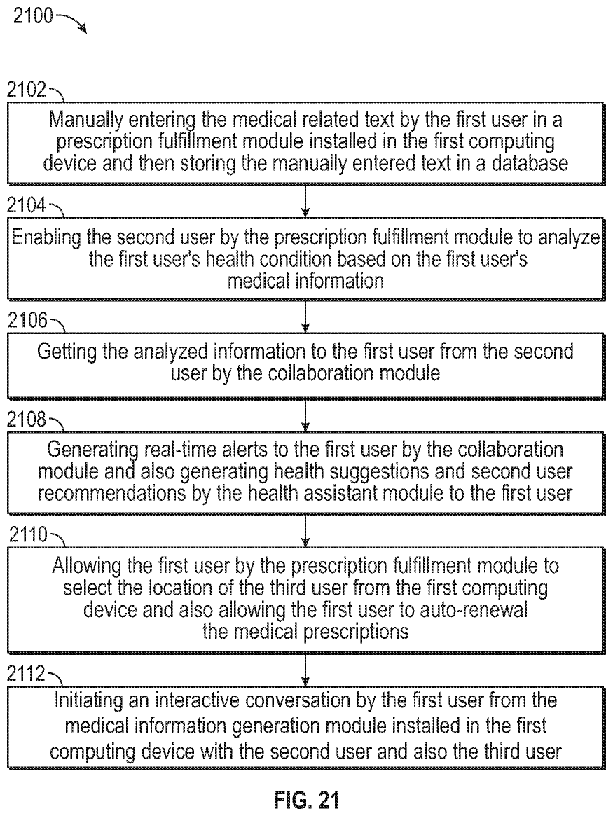

Referring to FIG. 21 is a flow chart 2100, depicting an exemplary method for collaborating the first user with the second user and the third user, in accordance with one or more exemplary embodiments. As an option, the method 1100 may be carried out in the context of the details of FIG. 1, FIG. 2, FIG. 3, FIG. 4, FIG. 5, FIG. 6, FIG. 7, FIG. 8, FIG. 9, FIG. 10, FIG. 11, FIG. 12, FIG. 13, FIG. 14, FIG. 15, FIG. 16, FIG. 17, and FIG. 18. FIG. 19, and FIG. 20. However, the method 2100 may also be carried out in any desired environment. Further, the aforementioned definitions may equally apply to the description below.

The first user manually enters the medical related text in the prescription fulfillment module through the first computing device and then stores the manually entered text in the database, at step 2104. Thereafter, at step 2104, the prescription fulfillment module enables the second user to analyze the first user's health condition based on the first user's medical information. Thereafter, at step 2106, the first user gets the analyzed information from the second user by the collaboration module. Thereafter, at step 2108, the collaboration module generates the real-time alerts to the first user and the health assistant module generates health suggestions and the second user recommendations to the first user. Thereafter, at step 2110, the prescription fulfillment module allows the first user to select the location of the third user from the first computing device and also allows the first user to auto-renew the prescriptions. Thereafter, at step 2112, the first user initiates the interactive conversation with the second user and also the third user from the medical information generation module installed in the first computing device.

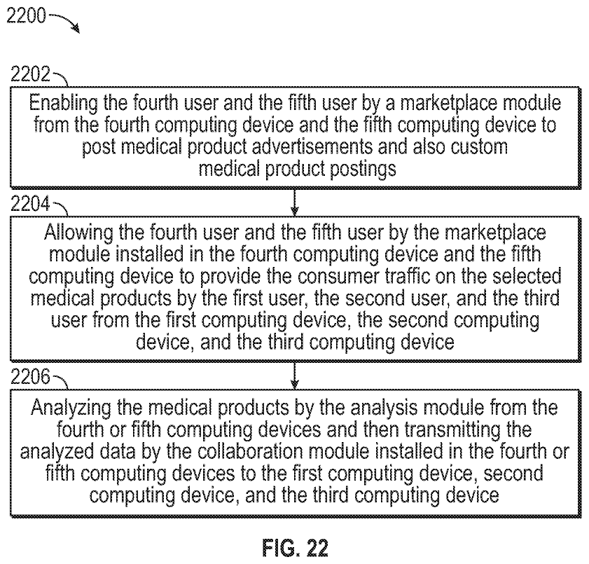

Referring to FIG. 22 is a flow chart 2200, depicting an exemplary method for collaborating the fourth user, fifth user with the first user, the second user, and the third user, in accordance with one or more exemplary embodiments. As an option, the method 2200 may be carried out in the context of the details of FIG. 1, FIG. 2, FIG. 3, FIG. 4, FIG. 5, FIG. 6, FIG. 7, FIG. 8, FIG. 9, FIG. 10, FIG. 11, FIG. 12, FIG. 13, FIG. 14, FIG. 15, FIG. 16, FIG. 17, and FIG. 18. FIG. 19, FIG. 20, and FIG. 21. However, the method 2200 may also be carried out in any desired environment. Further, the aforementioned definitions may equally apply to the description below.

The marketplace module enables the fourth user and the fifth user from the fourth computing device and the fifth computing device to post medical product advertisements and also custom medical product postings, at step 2202. Thereafter, at step 2204, the marketplace module allows the fourth user and the fifth user to provide the consumer traffic on the selected medical products by the first user, the second user, and the third user from the first computing device, the second computing device, and the third computing device. Thereafter, at step 2206, the analysis module analyzes the medical products from the fourth or fifth computing devices and then transmits the analyzed data by the collaboration module to the first computing device, the second computing device, and the third computing device from the fourth or fifth computing devices. Here, the analyzing medical products include artificial intelligence image to text conversion, cross-border medical product conversions, medical product conflicts, and user allergies.

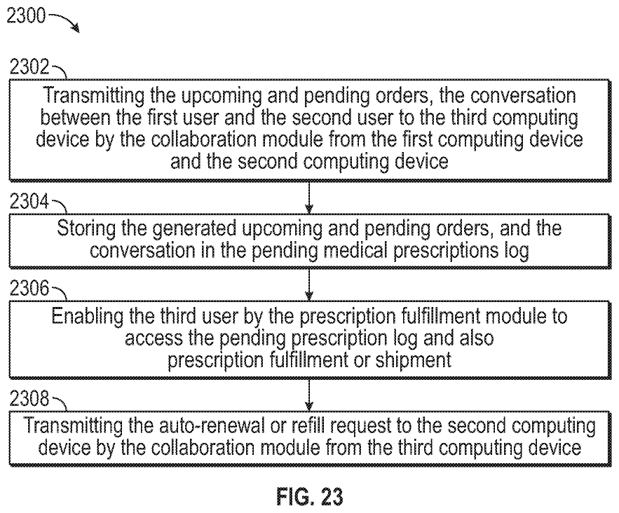

Referring to FIG. 23 is a flow chart 2300, depicting an exemplary method for collaborating the third user with other users, in accordance with one or more exemplary embodiments. As an option, the method 2300 may be carried out in the context of the details of FIG. 1, FIG. 2, FIG. 3, FIG. 4, FIG. 5, FIG. 6, FIG. 7, FIG. 8, FIG. 9, FIG. 10, FIG. 11, FIG. 12, FIG. 13, FIG. 14, FIG. 15, FIG. 16, FIG. 17, and FIG. 18. FIG. 19, and FIG. 20. However, the method 2300 may also be carried out in any desired environment. Further, the aforementioned definitions may equally apply to the description below.

The collaboration module from the first computing device and the second computing device transmits the upcoming and pending orders, the conversation between the first user and the second user to the third computing device, at step 2302. Thereafter, at step 2304, the pending medical prescriptions log stores the generated upcoming and pending orders, and the conversation. Thereafter, at step 2306, the prescription fulfillment module enables the third user to access the pending prescription log and also prescription fulfillment or shipment. Thereafter, at step 2308, the third computing device transmits the auto-renewal or refill request to the second computing device by the collaboration module.

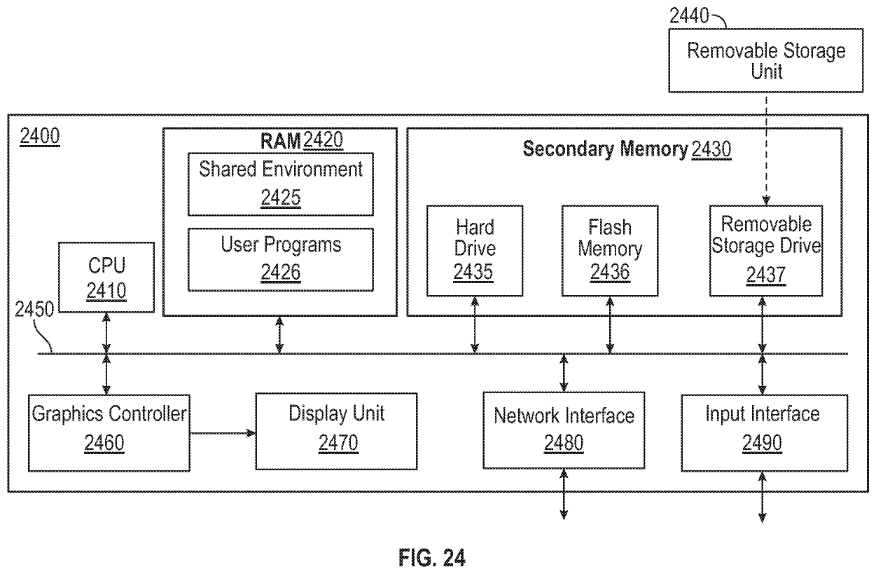

Referring to FIG. 24, FIG. 24 is a block diagram 2400 depicting the details of the computing device 2400 in which various aspects of the present disclosure are operative by execution of appropriate software instructions. The device may correspond to computing devices such as the first computing device 102/1002, the second computing device 104/1004, the third computing device 106/1006, the fourth computing device 108/1008, and the fifth computing device 110/1010 (or any other system in which the various features disclosed above can be implemented).

The end-user computing device 2400 may contain one or more processors such as a central processing unit (CPU) 2410, random access memory (RAM) 2420, secondary memory 2427, graphics controller 2460, display unit 2470, network interface 2480, and input interface 2490. All the components except display unit 2470 may communicate with each other over communication path 2450, which may contain several buses as is well known in the relevant arts. The components of FIG. 24 are described below in further detail.

CPU 2410 may execute instructions stored in RAM 2420 to provide several features of the present disclosure. CPU 2410 may contain multiple processing units, with each processing unit potentially being designed for a specific task. Alternatively, CPU 2410 may contain only a single general-purpose processing unit.

RAM 2420 may receive instructions from secondary memory 2430 using communication path 2450. RAM 2420 is shown currently containing software instructions, and/or data such as those used in threads and stacks, constituting shared environment 2425 and/or user programs 2426. Shared environment 2425 includes operating systems, device drivers, virtual machines, etc., which provide a (common) run time environment for execution of user programs 2426. The RAM 2420 may also be configured to store the modules of the medical information generation module 114.

Graphics controller 2460 generates display signals (e.g., in RGB format) to display unit 2470 based on data/instructions received from CPU 2410. Display unit 2470 contains a display screen to display the images defined by the display signals. Input interface 2490 may correspond to a keyboard and a pointing device (e.g., touch-pad, mouse) and may be used to provide inputs. The network interface 2480 provides connectivity to a network (e.g., using Internet Protocol), and may be used to communicate with other systems (such as those shown in FIG. 1), connected to the network 112.

Secondary memory 2430 may contain hard drive 2435, flash memory 2436, and removable storage drive 2437. Secondary memory 2430 may store the data software instructions (e.g., for performing the actions noted above with respect to the Figures), which enable digital processing system 2400 to provide several features in accordance with the present disclosure. The flash memory 2436 may include an electronic non-volatile computer storage medium that can be electrically erased and reprogrammed.

Some or all of the data and instructions may be provided on removable storage unit 2440, and the data and instructions may be read and provided by removable storage drive 2437 to CPU 2410. Floppy drive, magnetic tape drive, CD-ROM drive, DVD Drive, Flash memory, removable memory chip (PCMCIA Card, EEPROM) are examples of such removable storage drive 2437.

Removable storage unit 2440 may be implemented using medium and storage format compatible with removable storage drive 2437 such that removable storage drive 2437 can read the data and instructions. Thus, removable storage unit 2440 includes a computer readable (storage) medium having stored therein computer software and/or data. However, the computer (or machine, in general) readable medium can be in other forms (e.g., non-removable, random access, etc.).

In this document, the term “computer program product” is used to generally refer to removable storage unit 2440 or hard disk installed in hard drive 2435. These computer program products are means for providing software to digital processing system 2400. CPU 2410 may retrieve the software instructions, and execute the instructions to provide various features of the present disclosure described above.

The term “storage media/medium” as used herein refers to any non-transitory media that store data and/or instructions that cause a machine to operate in a specific fashion. Such storage media may comprise non-volatile media and/or volatile media. Non-volatile media includes, for example, optical disks, magnetic disks, or solid-state drives, such as storage memory 2430. Volatile media includes dynamic memory, such as RAM 2420. Common forms of storage media include, for example, a floppy disk, a flexible disk, hard disk, solid-state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge.

Storage media is distinct from but may be used in conjunction with transmission media. Transmission media participates in transferring information between storage media. For example, transmission media includes coaxial cables, copper wire and fiber optics, including the wires that comprise bus 2450. Transmission media can also take the form of acoustic or light waves, such as those generated during radio-wave and infra-red data communications.

Reference throughout this specification to “one embodiment”, “an embodiment”, or similar language means that a particular feature, structure, or characteristic described in connection with the embodiment is included in at least one embodiment of the present disclosure. Thus, appearances of the phrases “in one embodiment”, “in an embodiment” and similar language throughout this specification may, but do not necessarily, all refer to the same embodiment.

Although the present disclosure has been described in terms of certain preferred embodiments and illustrations thereof, other embodiments and modifications to preferred embodiments may be possible that are within the principles and spirit of the invention. The above descriptions and figures are therefore to be regarded as illustrative and not restrictive.

Thus the scope of the present disclosure is defined by the appended claims and includes both combinations and sub combinations of the various features described herein above as well as variations and modifications thereof, which would occur to persons skilled in the art upon reading the foregoing description.

What is claimed is:

1. An artificial intelligent platform for collaborating, automating and organizing health-information between users in the pharmaceutical/healthcare industries as well as users in the veterinarian space, comprising:
a medical information generation module configured to eliminate a tedious hard copy exchange and execution of a plurality of prescriptions by automating communication and fulfillment between a plurality of computing devices, whereby the plurality of computing devices configured to enable a plurality of users to access the plurality of prescriptions on the medical information generation module, the medical information generation module comprising:
a prescription fulfillment module configured to cover all steps of a typical prescription uses a medical information collected from a database to display different information on the plurality of computing devices based on the plurality of users regarding the fulfillment of the prescription, the prescription fulfillment module also comprises the automation of prescription handling by integrating with a plurality of healthcare/pharmacy/veterinary systems to provide a secure and streamlined communication system;
a collaboration module configured to collaborate the plurality of users with each other comprising of a direct communication chat function and also configured to generate a plurality of real time alerts to proper users on the plurality of computing devices for a plurality of reminders such as prescription refill reminders and updates, key health check recommendation reminders, and due lab work reminders;
a health assistant module configured to provide a plurality of customized suggestions and recommendations from artificial intelligence models on the plurality of computing devices, the health assistant module configured to communicate with the collaboration module and a marketplace module to display prescription information, stakeholder information, patient information and history, a plurality of products and a plurality of offers on the plurality of computing devices;
an analytics module configured to convert a plurality of images to text using artificial intelligence techniques on the plurality of computing devices, the text is used to capture drug information and correlations, and cross border drug conversions;
a marketplace module configured to provide easy access for the plurality of users to view, purchase and receive the plurality of items such as drugs, supplements, vitamins, hygienic products, Ayurveda, health related services, yoga, consultations, and life coaches services, the marketplace module configured to allow a plurality of drug manufacturers, supplements providers and service providers to display, sell and control products they offer using artificial intelligence, product and services recommendations based on past prescriptions and purchases;
a virtual pharmacy and consultation module configured to provide a virtual pharmacy that enables the plurality of users on the plurality of computing devices to purchase medicine and drug/supplements virtually based on the plurality of prescriptions as well as view videos and have video consultations with the plurality of users; and
a prescription auction module configured to allow a plurality of first users to upload the plurality of prescriptions and consultation services to an auction post on the plurality of computing devices, a plurality of second users able to view and bid the auction post on the plurality of computing devices and then fulfil the plurality of prescriptions or required services after winning an auction.

2. The artificial intelligent platform of claim 1, wherein the prescription fulfillment module is configured to convert prescription data from an image and using artificial intelligence detect/understand the information within a typical patient/veterinarian prescription, including patient details, drug name and amounts, date and time of assigned prescription, and drug intake instructions.

3. The artificial intelligence platform of claim 1, wherein the prescription fulfillment module is configured to integrate with the plurality of healthcare/pharmacy/hospital/medical/veterinarian systems.

4. The artificial intelligent platform of claim 1, wherein the collaboration module is configured to provide a common cloud platform in which the plurality of first users are directly communicate and collaborate with the plurality of second users and a plurality of third users.

5. The artificial intelligent platform of claim 1, wherein the collaboration module is configured to allow the easy collaboration of a plurality of services by providing an easy way to execute the plurality of services and functionality provided by the artificial intelligent platform including, prescription corrections, prescription refills, supplement recommendations, and links to specific auction posts.

6. The artificial intelligent platform of claim 1, wherein the collaboration module is configured to send the plurality of real time alerts between the plurality of first users, the plurality of second users, the plurality of third users, a plurality of fourth users, and a plurality of fifth users.

7. The artificial intelligent platform of claim 1, wherein the analytics module is configured to send the plurality of first users with information and a plurality of insights on a plurality of drugs.

8. The artificial intelligent platform of claim 1, wherein the marketplace module is configured to host a safe and secure marketplace for the plurality of users to purchase and list drugs, supplements, and a natural medicine.

9. The artificial intelligent platform of claim 1, wherein the virtual pharmacy and consultation module is configured to allow the plurality of second users to host pharmacies virtually.

10. The artificial intelligent platform of claim 1, wherein the virtual pharmacy and consultation module is configured to have an interface to host custom instructional videos based on the plurality of prescriptions.

11. The artificial intelligent platform of claim 1, wherein the virtual pharmacy and consultation module is configured to host their practices virtually and have virtual video consultation functions for the plurality of second users.

12. The artificial intelligent platform of claim 1, wherein the prescription auction module is configured to allow the plurality of first users to post prescriptions and services up for bidding/auction.

13. The artificial intelligent platform of claim 1, wherein the health assistant module is configured to provide the plurality of customized suggestions from artificial intelligence models and also configured access other modules to give the user easy access to trigger services.

14. The artificial intelligent platform of claim 1, wherein the prescription fulfillment module is configured to display a plurality of pending prescriptions, prescriptions are currently in use, what prescriptions are completed, pending auto-renewals, and prescription progress where there is need to have an access and to visualize on the plurality of computing devices.

15. The artificial intelligent platform of claim 1, wherein the prescription fulfillment module is also configured to display the plurality of first users to the plurality of second users that they are dealing with along with each user's current and completed prescriptions on the plurality of computing devices.

16. The artificial intelligent platform of claim 1, wherein the health assistant module comprises a correlation and conflict module configured to correlate and conflict the medical information in the plurality of prescriptions and transmit the plurality of real time alerts through the collaboration module.

17. The artificial intelligent platform of claim 1, wherein the medical information generation module is configured to provide the medical information such as past medical history, family medical history, medication history, personal and lifestyle history, psychological analysis, vital signs, electrocardiogram (EKG or ECGs), and other lab reports such as drug level, bacteria in urine, bacteria in the blood, activities of the individual, and glucose levels.

18. The artificial intelligent platform of claim 1, wherein the medical information generation module is configured to provide veterinary medical information such as past medical history animals, breed medical history, medication history, personal and lifestyle history of animals, psychological analysis, vital signs, electrocardiogram (EKG or ECGs), and other lab reports such as drug level, bacteria in urine, bacteria in the blood, activities of the individual, and glucose level.

19. A method for collaborating and organizing medical information between multiple users in the pharmaceutical/healthcare industries, comprising:
enabling a plurality of users to login to a medical information generation module by creating user accounts using login identity credentials through a plurality of computing devices, whereby the medical information generation module configured to provide a plurality of custom webpages based on the type of a user and execution of a plurality of prescriptions by automating communication and fulfillment between the plurality of computing devices;
enabling the plurality of users to access a plurality of medical prescriptions on the medical information generation module, whereby the plurality of medical prescriptions are securely stored in a database;
collaborating the plurality of users with each other and also generating a plurality of real time alerts to proper users on the plurality of computing devices for prescription refill reminders, key health check recommendation reminders, and due lab work reminders;
generating medical related suggestions and recommendations using artificial intelligence on the plurality of computing devices by the medical information generation module; and
providing a marketplace to a plurality of first users, a plurality of second users, and a plurality of third users on the plurality of computing devices through the medical information generation module and an easy access to product purchases from a plurality of fourth users and a plurality of fifth users.

20. A computer program product comprising a non-transitory computer-readable medium having a computer-readable program code embodied therein to be executed by one or more processors, said program code including instructions to:
enable a plurality of users to login to a medical information generation module by creating user accounts using login identity credentials through a plurality of computing devices, whereby the medical information generation module configured to provide a plurality of custom webpages based on a type of the user and execution of a plurality of prescriptions by automating communication and fulfillment between the plurality of computing devices;
enable the plurality of users to access a plurality of medical prescriptions on the medical information generation module, whereby the plurality of medical prescriptions are securely stored in a database;
collaborate the plurality of users with each other and also generating a plurality of real time alerts to proper users on the plurality of computing devices for prescription refill reminders, key health check recommendation reminders, and due lab work reminders;
generate medical related suggestions and recommendations using artificial intelligence on the plurality of computing devices by the medical information generation module; and
provide a marketplace to a plurality of first users, a plurality of second users, and a plurality of third users on the plurality of computing devices through the medical information generation module and an easy access to product purchases from a plurality of fourth users and a plurality of fifth users.
